(19) United States
(12) Patent Application Publication
(10) Pub. No.: US 2010/0190180 A1
(43) Pub. Date: Jul. 29, 2010

(54) Materials and Methods for Screening, Diagnosis and Prognosis of Conditions Associated With Stat Protein Expression

(76) Inventor: Heidi Kay, Springfield, VA (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/988,315

(22) PCT Filed: Jul. 6, 2006
(86) PCT No.: PCT/US2006/026401

§ 371 Date: Feb. 16, 2010

Related U.S. Application Data
(60) Provisional application No. 60/696,742, filed on Jul. 6, 2005.

(51) Int. Cl. G01N 33/53; C07F 15/00

(52) U.S. Cl. 435/7.1; 544/225; 556/137; 556/7; 549/212; 548/402; 548/101; 536/121

(57) ABSTRACT

The subject invention concerns methods and materials for cancer screening using platinum complexes to detect a STAT protein biomarker. Platinum (IV) complexes interacting with STATs directly correlate with the STAT expression. In one embodiment, fluorescently-labeled and/or antibody-linked platinum (IV) complexes can be used to assess the STAT expression and define malignant potential. Other methods such as imaging (MRI, e.g.) can also be used to assess platinum-STAT interactions. The STAT protein can be, for example, STAT3.

### CROSS-REFERENCE TO A RELATED APPLICATION

This application claims the benefit of U.S. Provisional Application Ser. No. 60/696,742, filed Jul. 6, 2005, which is hereby incorporated by reference herein in its entirety, including any figures, tables, and drawings.

### BACKGROUND OF THE INVENTION

Cellular responses to growth factors and cytokines are characterized by activation of the Signal Transducer and Activator of Transcription (STAT) family of cytoplasmic transcription factors (Darnell, 1997; Darnell et al., 1994; Schindler et al., 1995; Stark et al., 1998; Smithgall et al., 2000; Akira, 2000; Hirano et al., 2000; Bromberg et al., 1996; Fukada et al., 996; Kotenko et al., 2000). STATs are activated at a very early stage in the transduction pathway by tyrosine phosphorylation that is induced by protein tyrosine kinases of growth factor receptors, receptor-associated Janus kinase (Jaks) or Src kinase families. This turn induces phosphotyrosine (pTyr)-SH2 interactions between two STAT monomers and the formation of dimers, which then translocate to the nucleus, bind to specific DNA response elements and regulate the expression of genes essential for cell proliferation, differentiation, development and survival.

Normal STAT activation is tightly-regulated and has a short duration, which is in keeping with normal cellular requirements for mounting a response to external stimuli. However, persistent activation of specific STAT proteins, particularly Stat3 and Stat5, occurs with high frequency in some tumors, and persistently-active Stat3 has a causal role in malignant transformation by promoting growth and survival of transformed and tumor cells, including those of breast, prostate and head and neck squamous carcinoma cells, lymphomas and leukemias (Bromberg et al., 1999; Turkson et al., 1998; Bromberg et al., 1998; Catlett-Falcone et al., 1999a; Garcia et al., 2001; Grandis et al., 2000a; Grandis et al., 1998; Nielsen et al., 1997; Nielsen et al., 1999; Epling-Burnette et al., 2001; reviewed in Bowman et al., 2000a; Turkson et al., 2000; Song et al., 2000; Coffer et al., 2000; Lin et al., 2000; Catlett-Falcone et al., 1999b; Garcia et al., 1998). Of clinical importance, blockade of Stat3 signaling in malignant cells or whole tumors that containing persistently-activated Stat3 induces apoptosis and tumor regression.

Platinum complexes, the prototype of cisplatin, have been widely used as active anticancer agents (Ardizzoni et al., 1999; Nitiss, 2002) in a variety of human tumors, including testicular, ovarian, bladder carcinoma, head and neck, and non-small cell lung cancers. The outcome of treatments with cisplatin and other platinum-containing compounds is strongly linked to their alkylating effects on DNA. However, the potential impact of platinum-complex-based therapy on cellular signaling and the therapeutic importance of such interactions have yet to be explored. Reports show that cisplatin induces activation of members of the mitogen-activated protein kinase (MAPK) pathways (Persons et al., 1999; Sanchez-Perez et al., 1998), which may influence drug-induced apoptosis.

Histological screening for cancer cells in cell culture is unreliable. Protein biomarkers for early detection of cancers are anticipated to transform diagnosis. Detection of a biomarker at low concentrations amidst a myriad of proteins is, however, a limitation of this technology. Additionally, identification of a common protein screen for multiple cancer lines is clearly advantageous.

This work proposes targeting STAT proteins, such as STAT3, which are specifically upregulated in diverse human tumors and overexpressed in precancerous cells, with fluorescently labeled small-molecule inhibitors of STAT proteins. Clinical screening of cells in tissue culture will then provide preliminary diagnosis. Furthermore, STAT3 proteins can be selectively identified, quantified and characterized by techniques such as flow cytometry, quantitative RT-PCR or solid phase microextraction coupled with capillary isoelectric focusing and laser-induced fluorescence. High levels of STAT3 are associated with more aggressive and metastatic disease-recognition of which is critical to prescribed treatments. Lastly, development of antibody-linked and biopolymer-coated nanoparticles composed of these same small-molecule STAT3 inhibitors will both facilitate efficient diagnosis and potential tailored treatments of characterized STAT3 expressions.

### BRIEF SUMMARY OF THE INVENTION

The subject invention concerns methods and materials for screening for conditions associated with STAT protein expression using platinum complexes as a STAT protein biomarker. Platinum (IV) complexes interacting with STATs directly correlate with the STAT expression. In one embodiment, platinum (IV) complexes comprising a detectable label can be used to assess the STAT expression and define malignant potential. Other methods, such as radiographic, scintigraphic and magnetic resonance imaging, can also be used to assess platinum-STAT interactions. The STAT protein can be, for example, STAT3.

### BRIEF DESCRIPTION OF THE FIGURES

FIGS. 1A-1B are photographs of Murine Pancreatic H2 cells that express low levels of STAT3. The cells were incubated with 25 μM platinum (IV) complex (designated herein as “CPA51”) comprising a luminol substituent for 36 hours. Images are under fluorescence microscopy (FIG. 1A) and light microscopy (FIG. 1B) at 640× magnification.

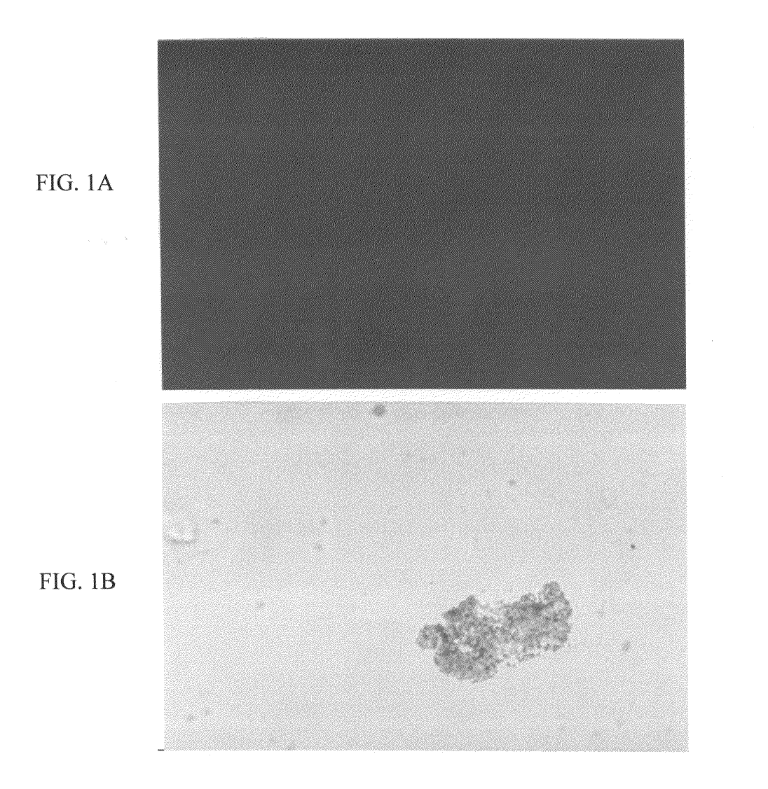

FIGS. 2A-2B are photographs of Murine Pancreatic H2 cells that express low levels of STAT3. The cells were incubated with 25 μM platinum (IV) complex (designated herein as “CPA51”) comprising a luminol substituent for 36 hours. Images are under light microscopy (FIG. 2A) and fluorescence microscopy (FIG. 2B) at 400× magnification.

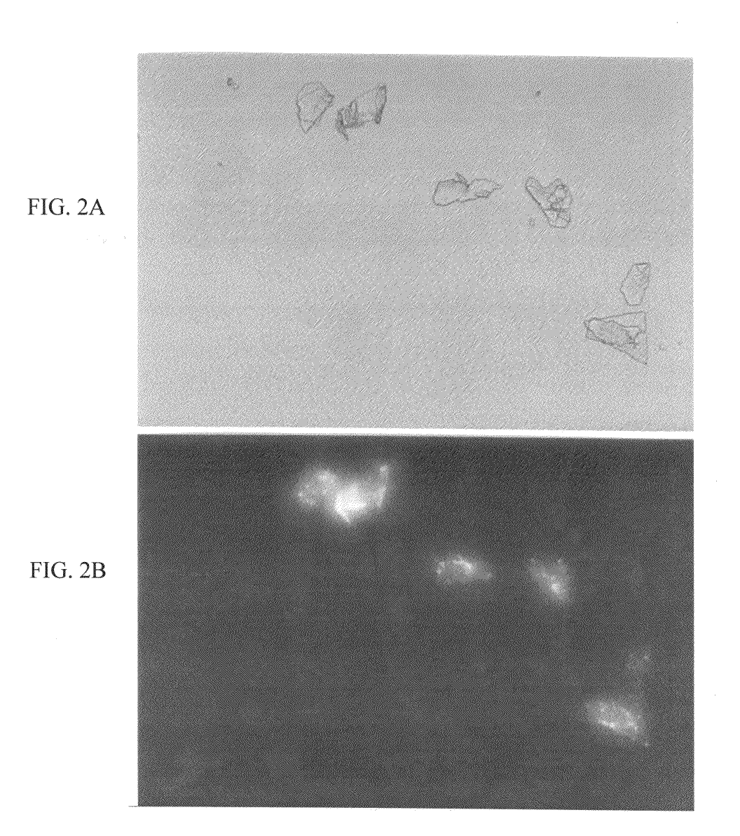

FIGS. 3A-3B are photographs of Murine Pancreatic H2 cells that express low levels of STAT3. The cells were incubated with 25 μM platinum (IV) complex (designated herein as “CPA51”) comprising a luminol substituent for 36 hours. Images are under light microscopy (FIG. 3A) and fluorescence microscopy (FIG. 3B) at 400× magnification.

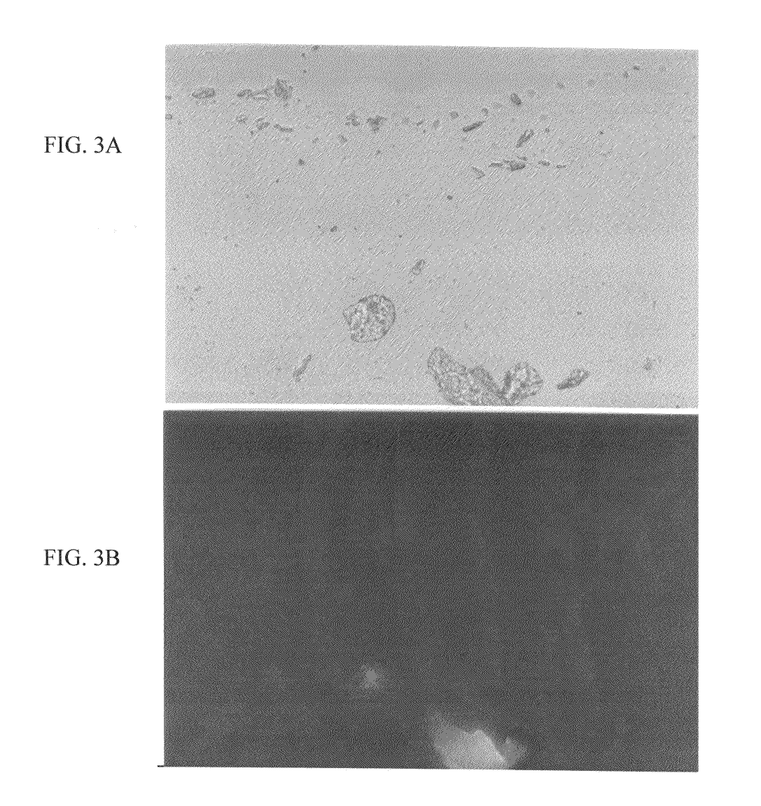

FIGS. 4A-4B are photographs of Murine Pancreatic H7 cells that express high levels of STAT3. The cells were incubated with 25 μM platinum (IV) complex (designated herein as “CPA51”) comprising a luminol substituent for 36 hours. Images are under light microscopy (FIG. 4A) and fluorescence microscopy (FIG. 4B) at 400× magnification.

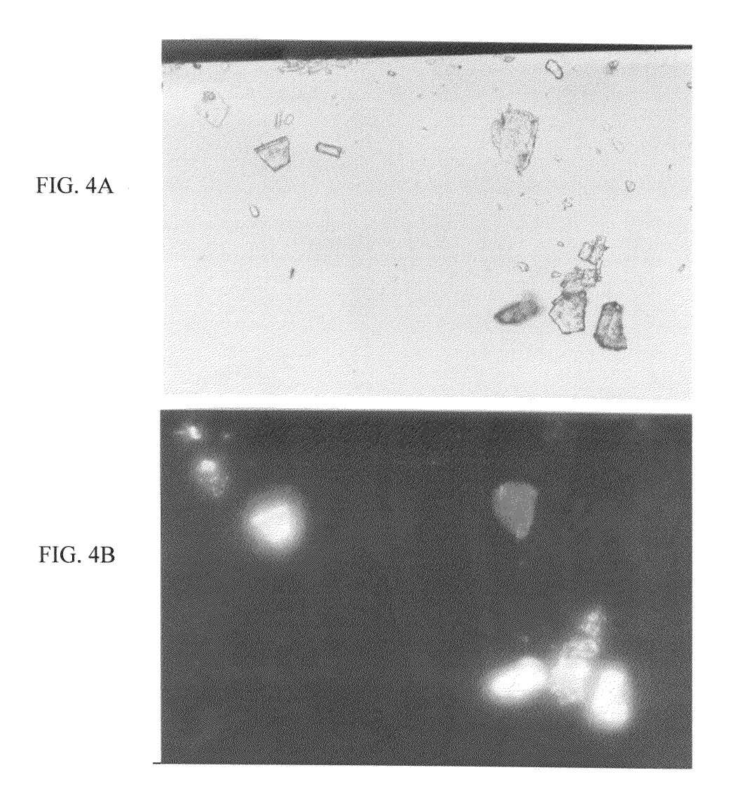

FIGS. 5A-5B are photographs of Murine Pancreatic 117 cells that express high levels of STAT3. The cells were incubated with 25 μM platinum (IV) complex (designated herein as “CPA51”) comprising a luminol substituent for 36 hours. Images are under light microscopy (FIG. 5A) and fluorescence microscopy (FIG. 5B) at 400× magnification.

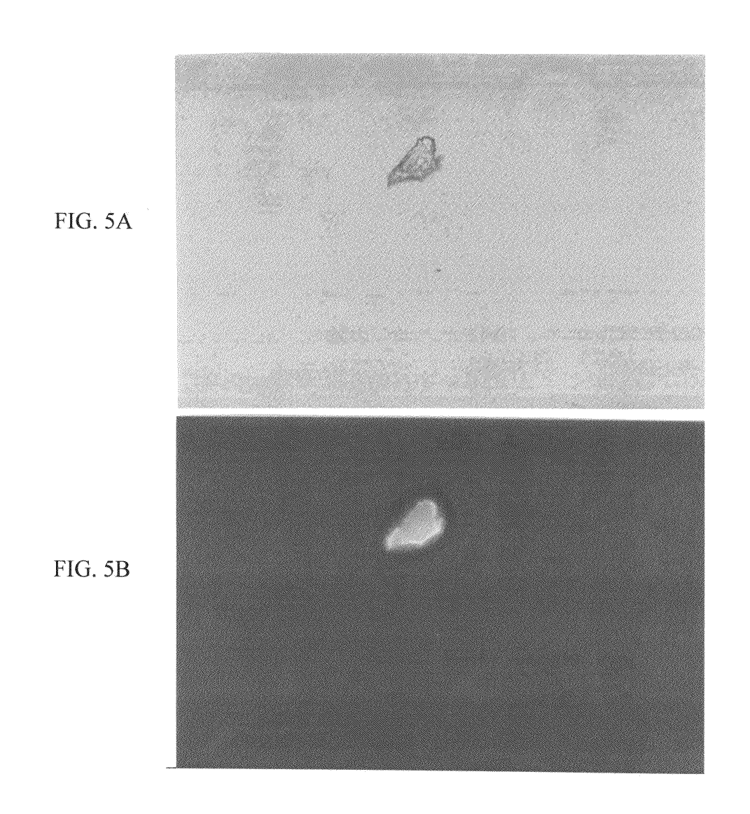

FIGS. 6A-6B are photographs of Murine Pancreatic H7 cells that express high levels of STAT3. The cells were incubated with 25 μM platinum (IV) complex (designated herein as “CPA51”) comprising a luminol substituent for 36 hours. Images are under light microscopy (FIG. 6A) and fluorescence microscopy (FIG. 6B) at 400× magnification.

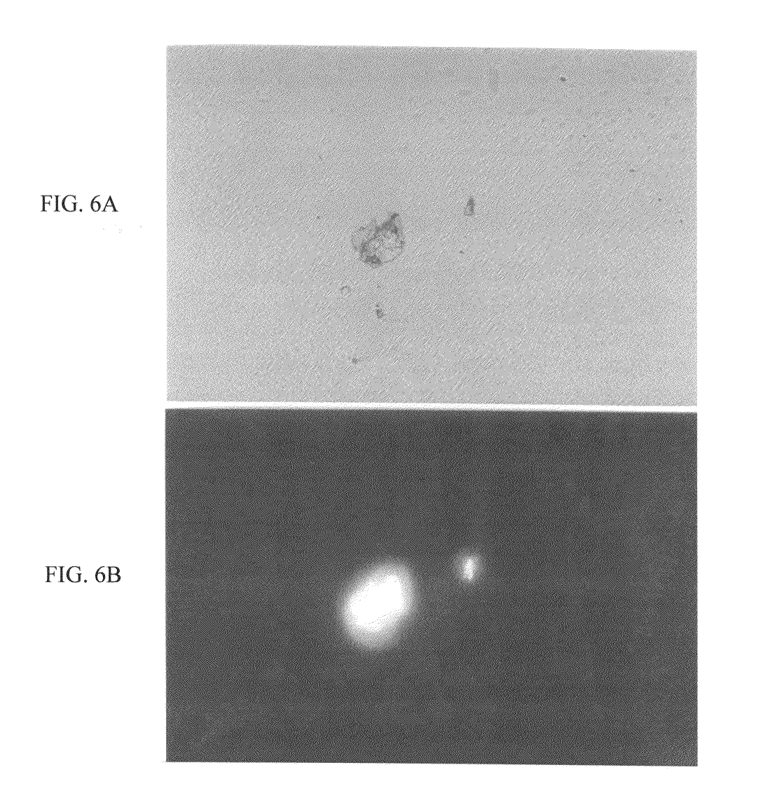

FIGS. 7A-7B are photographs of Murine Pancreatic H7 cells that express high levels of STAT3. The cells were incubated with 25 μM platinum (IV) complex (designated herein as “CPA51”) comprising a luminol substituent for 36 hours. Images are under light microscopy (FIG. 7A) and fluorescence microscopy (FIG. 7B) at 400× magnification.

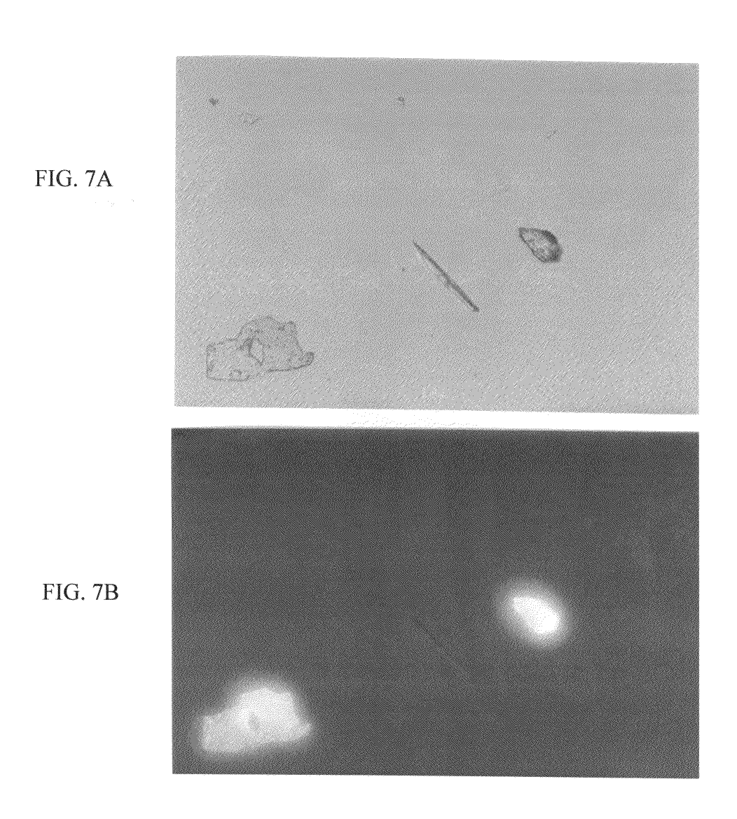

### DETAILED DISCLOSURE OF THE INVENTION

The subject invention concerns methods and materials for screening for conditions associated with abnormal levels of expression of a STAT protein, such as STAT3, using a platinum (IV) complex. The upregulation of STAT3 proteins in over 85% of cancerous cells identifies an intracellular protein biomarker useful for early detection, characterization and treatment of multiple cancers including, but not limited to, breast cancers, prostate cancers, head and neck cancers, lymphomas and leukemias, melanomas, colon cancers, and lung cancers. Platinum (IV) complexes useful in the present invention are small-molecule inhibitors of STAT3 that have demonstrated marked success both in vitro and in vivo (Turkson et al., 2004). Platinum complexes useful in the invention, such as the platinum complexes designated herein as CPA-1 and CPA-7, physically interact with the DNA-binding domain and/or the phosphorylation of Stat3 proteins. Src-transformed mouse fibroblasts, as well as human tumor cells of the breast, prostate, and lung, and mouse melanoma cells contain constitutive Stat3 activity. Thus, the presence of STAT protein is a biomarker for a cancerous or neoplastic condition, as well as other conditions, which can be detected by screening for uptake of a platinum complex according to the present invention.

In one embodiment, platinum (IV) complexes, for example, a detectably labeled platinum (IV) complex, provide a simple in vitro early screening tool with the advantage of assessing treatment diagnosis and prognosis. Additional measurements of intracellular STAT protein biomarker expression using for example, flow cytometry, quantitative RT-PCR, etc. can be made to establish suitable applications for optimum therapeutic treatment as well as establishing verification and characterization of anomalies (Irish et al. (2004); Krutzik et al. (2004)). For example, if using flow cytometry, phosphorylation states of STAT proteins are labeled with phospho-specific antibodies against selected epitopes. Cells are stimulated, fixed, permeabilized and stained with respective antibodies. Data is collected for each cell and can be correlated with surface marker expressions, with simultaneous parameters possible for each cell. Western Blotting and ELISA can also be used to determine relative STAT expressions. Development of antibody-linked and biopolymer-coated nanoparticles composed of platinum (IV) complexes that interacting with STAT proteins also facilitate both efficient diagnosis and potential tailored treatments of characterized STAT expression.

Platinum complexes useful in the present invention include those complexes having the structure shown in formula IA or IB:

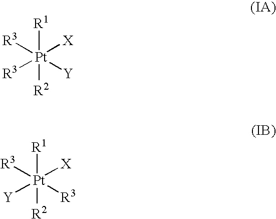

wherein
X and Y are, independently, any halogen, —NO₂, —ONO, or the structure:

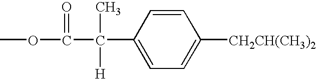

or X and Y together form the structure:

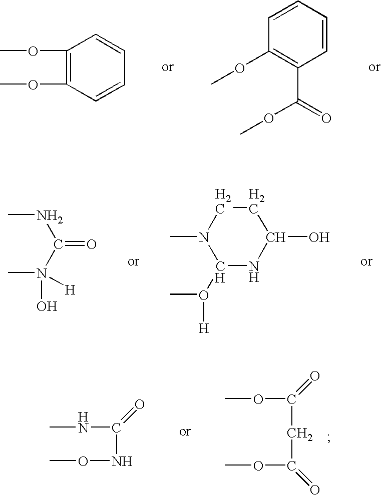

### R¹ is —NO₂, —ONO, Cl, Br or F;

R² is any halogen, —OH, —ONO, —ONO₂, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure:

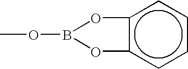

any of which can be substituted with any halogen, —NH₂, —COOH, alkoxy, cycloalkoxy;
R³ is, independently, —NH₃, —NHR⁷, —NH₂R⁷, —NH(R⁷)₂, or —N(R⁷)₃;
R⁷ is H, C₁₋₆ alkyl, alkoxy, or aryl, any of which can be optionally substituted with any halogen, —NO₂, or —COOH;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
or a pharmaceutically acceptable salt thereof.

In one embodiment, X and Y can be, independently, fluorine (F), chlorine (Cl), bromine (Br) or iodine (I). In an exemplified embodiment, X is Cl and Y is Cl.

In one embodiment, R¹ is —NO₂, R² is Cl and R³ is —NH₃.

In one embodiment, a compound of formula IA or IB has as an R² substituent any of the axial ligands attached to the platinum atom of the platinum complexes of Table 1. In one exemplified embodiment, a compound of the invention has the chemical structure shown for the compound designated as CPA51 shown in Table 1.

Platinum complexes of the invention can also have the structure shown in formula II:

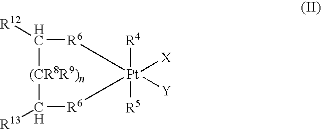

wherein
X and Y are, independently, any halogen, or the structure:

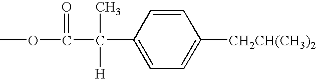

or X and Y together form the structure:

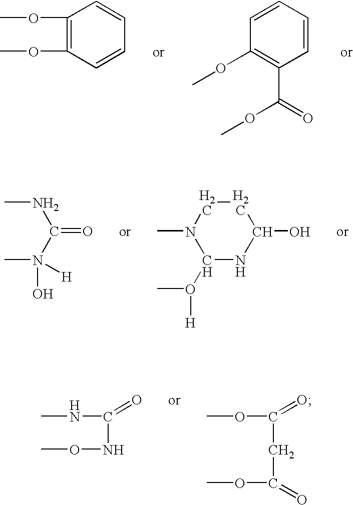

### R⁴ is —NO₂ or —ONO;

R⁵ is any halogen, —OH, —ONO, —ONO₂, —COR¹⁰, —OPO₃R¹⁰R¹¹, —OSO₃H, —OSeOOH, SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure:

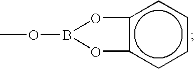

any of which can be substituted with any halogen, —NH₂, —COOH, —OH, or Y and R⁵ form the structure:

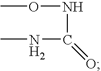

or X and Y together form the structure:

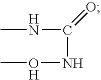

R⁶ is, independently, NH₂, NH, NHR⁷, N(R⁷)₂, NHR⁸, N(R⁸)₂, NHR⁹, N(R⁹)₂, or NR⁸R⁹;
R⁷ is H, C₁₋₆ alkyl, alkoxy, aryl, any of which can be optionally substituted with any halogen, —NO₂, or —COOH;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H or C₁₋₆ alkyl, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof.

In one embodiment, X and Y can be, independently, fluorine (F), chlorine (Cl), bromine (Br) or iodine (I). In an exemplified embodiment, X is Cl and Y is Cl.

In one embodiment, R⁴ is —NO₂, R⁵ is Cl, R⁶ is —NH₂, and n is 0.

In one embodiment, a compound of formula II has as an R⁵ substituent any of the axial ligands attached to the platinum atom of the platinum complexes of Table 1.

Platinum complexes of the invention can also have the structure shown in formula III or formula IVA or IVB:

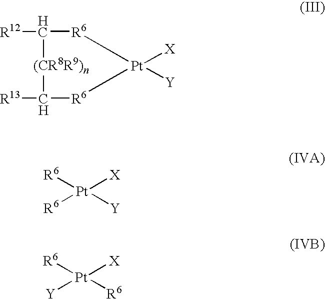

wherein
X and Y are, independently, any halogen, —NO₂, —ONO, or X and Y together form the structure:

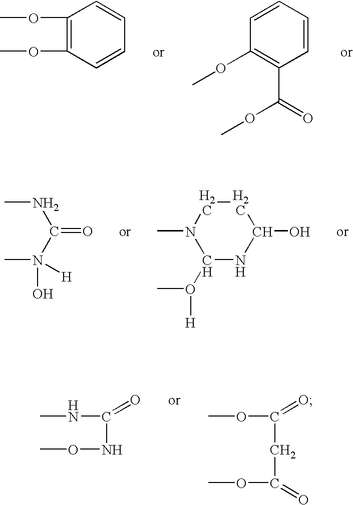

R⁶ is, independently, Cl, Br, F, NO₂, ONO, NHR⁸, NH₂, NHR¹², NR¹², N(R¹²)₂, NHR¹³, NR¹³, N(R¹³)₂, NR¹²R¹³;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H, C₁₋₆ alkyl, or —OH, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof.

In one embodiment, X and Y can be, independently, fluorine (F), chlorine (Cl), bromine (Br) or iodine (I). In an exemplified embodiment, X is Cl and Y is Cl.

Also contemplated within the scope of the invention are platinum complexes that are not defined by formula IA or IB or formula II but that are specifically exemplified in the Table 1 presented herein. Exemplified embodiments of platinum complexes of the invention are shown in Table 1. The chemical structure of a complex along with a designation name (e.g., CPA-XX) is shown in the Table. Alternative designation names (e.g., HKXXX) of a complex are shown in parentheses.

Platinum complexes of the invention also include those complexes having the structure shown in formula VA or VB or formula VI:

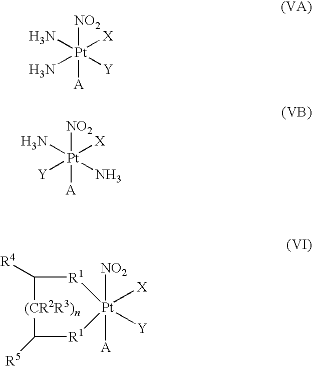

wherein
X and Y are, independently, any halogen, —OH, H₂O, or —SO(CH₃)₂;
or X and Y together form the structure:

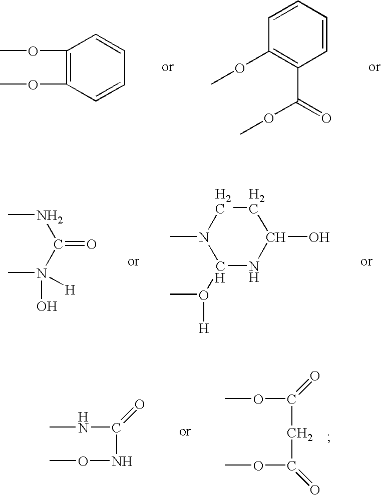

and A can be any of the following:

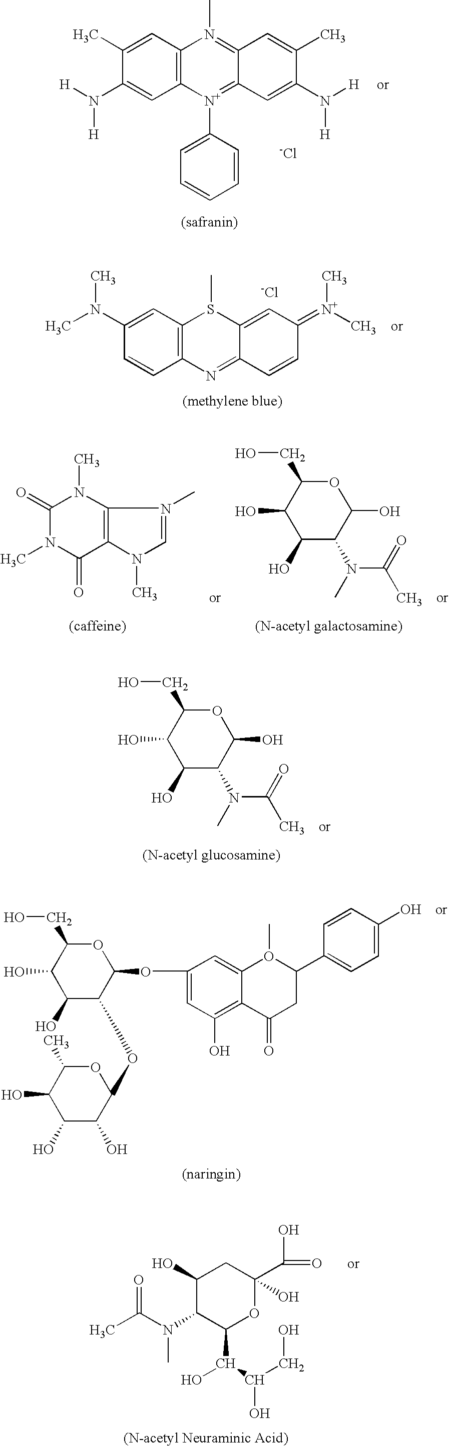

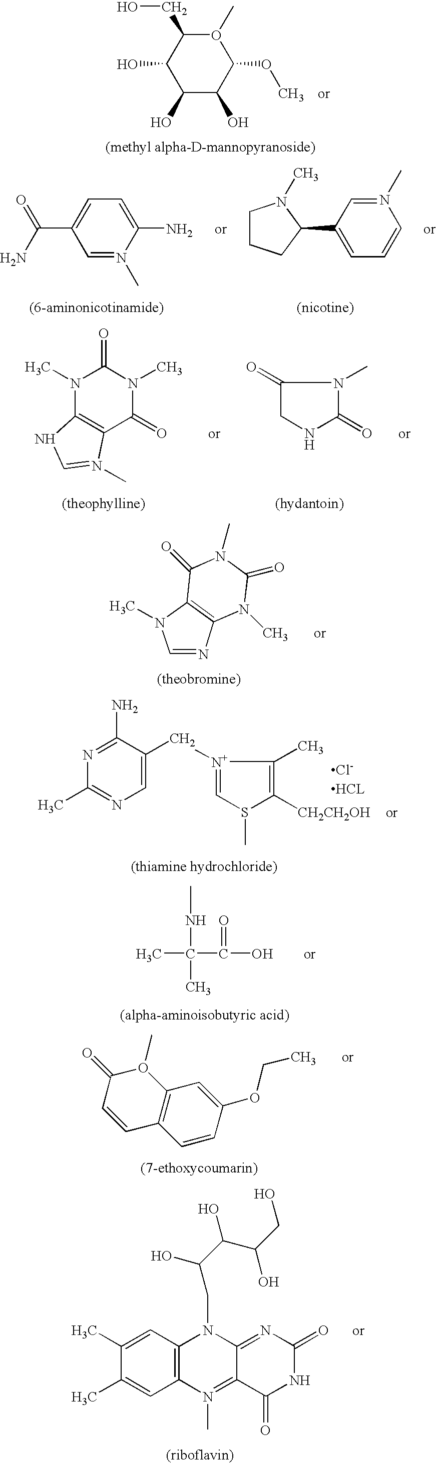

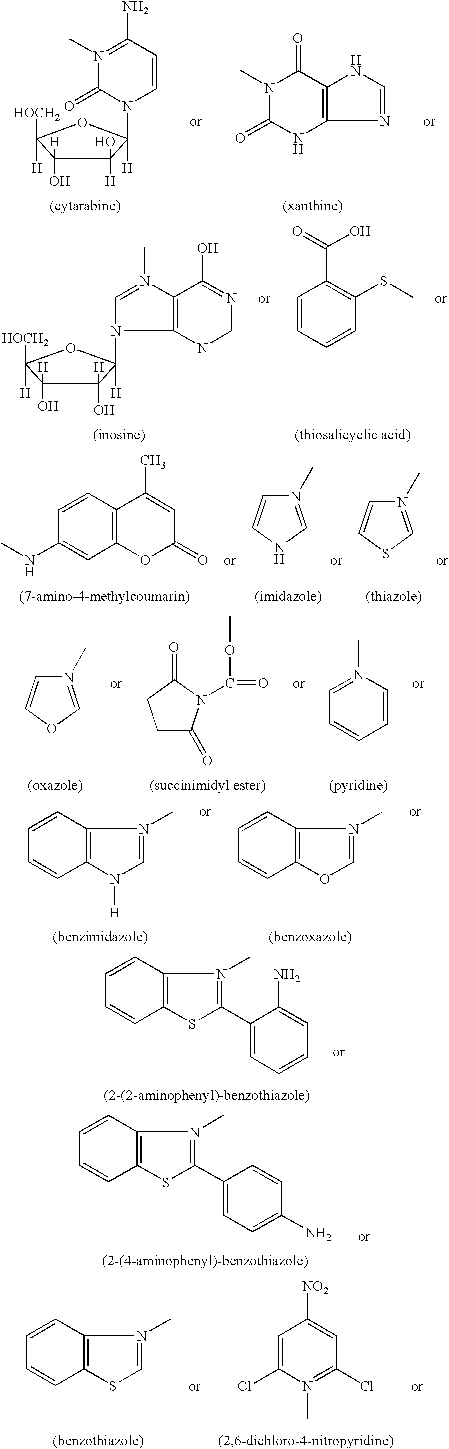

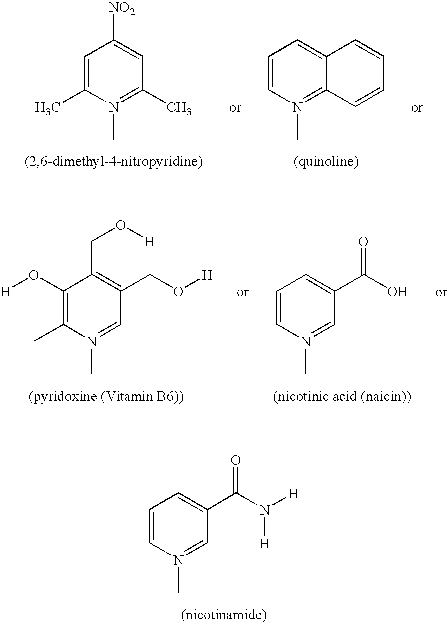

and wherein
R¹ is, independently, NH₂, NH, NR⁴, NHR⁴, N(R⁴)₂, NR⁵, NHR⁵, N(R⁵)₂, or NR⁴R⁵;
R² and R³ are, independently, H, —OH, C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
R⁴ and R⁵ are, independently, H or C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl or R⁴ and R⁵ together form a cycloalkyl, cycloalkoxy, aryl, aryloxy, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
n is any integer from 0 to 6;
or a pharmaceutically acceptable salt thereof.

In one embodiment, X and Y can be, independently, chlorine (Cl), bromine (Br) or iodine (I). In an exemplified embodiment, X is Cl and Y is Cl.

As used herein, alkyl means straight or branched chain, saturated or mono- or polyunsaturated hydrocarbon groups having from 1 to 20 carbon atoms and C_{1-X} alkyl means straight or branched chain alkyl groups containing from one up to X carbon atoms. For example, C₁₋₆ alkyl means straight or branched chain alkyl groups containing from one up to 6 carbon atoms. Alkoxy means an alkyl-O— group in which the alkyl group is as previously described. Cycloalkyl includes a nonaromatic monocyclic or multicyclic ring system, including fused and spiro rings, of from about three to about 10 carbon atoms. A cyclic alkyl may optionally be partially unsaturated. Cycloalkoxy means a cycloalkyl-O— group in which cycloalkyl is as defined herein. Aryl means an aromatic monocyclic or multicyclic carbocyclic ring system, including fused and spiro rings, containing from about six to about 14 carbon atoms. Aryloxy means an aryl-O— group in which the aryl group is as described herein. Alkylcarbonyl means a RC(O)— group where R is an alkyl group as previously described. Alkoxycarbonyl means an ROC(O)— group where R is an alkyl group as previously described. Cycloalkylcarbonyl means an RC(O)— group where R is a cycloalkyl group as previously described. Cycloalkoxycarbonyl means an ROC(O)— group where R is a cycloalkyl group as previously described.

Heteroalkyl means a straight or branched-chain having from one to 20 carbon atoms and one or more heteroatoms selected from nitrogen, oxygen, or sulphur, wherein the nitrogen and sulphur atoms may optionally be oxidized, i.e., in the form of an N-oxide or an S-oxide. Heterocycloalkyl means a monocyclic or multicyclic ring system (which may be saturated or partially unsaturated), including fused and spiro rings, of about five to about 10 elements wherein one or more of the elements in the ring system is an element other than carbon and is selected from nitrogen, oxygen, silicon, or sulphur atoms. Heteroaryl means a five to about a 14-membered aromatic monocyclic or multicyclic hydrocarbon ring system, including fused and spiro rings, in which one or more of the elements in the ring system is an element other than carbon and is selected from nitrogen, oxygen, silicon, or sulphur and wherein an N atom may be in the form of an N-oxide. Arylcarbonyl means an aryl-CO-group in which the aryl group is as described herein. Heteroarylcarbonyl means a heteroaryl-CO-group in which the heteroaryl group is as described herein and heterocycloalkylcarbonyl means a heterocycloalkyl-CO-group in which the heterocycloalkyl group is as described herein. Aryloxycarbonyl means an ROC(O)-group where R is an aryl group as previously described. Heteroaryloxycarbonyl means an ROC(O)-group where R is a heteroaryl group as previously described. Heterocycloalkoxy means a heterocycloalkyl-O-group in which the heterocycloalkyl group is as previously described. Heterocycloalkoxycarbonyl means an ROC(O)-group where R is a heterocycloalkyl group as previously described.

Examples of saturated alkyl groups include, but are not limited to, methyl, ethyl, N-propyl, isopropyl, N-butyl, tert-butyl, isobutyl, sec-butyl, N-pentyl, N-hexyl, N-heptyl, and N-octyl. An unsaturated alkyl group is one having one or more double or triple bonds. Unsaturated alkyl groups include, for example, ethenyl, propenyl, butenyl, hexenyl, vinyl, 2-propynyl, 2-isopentenyl, 2-butadienyl, ethynyl, 1-propynyl, 3-propynyl, and 3-butynyl. Cycloalkyl groups include, for example, cyclopentyl, cyclohexyl, 1-cyclohexenyl, 3-cyclohexenyl, and cycloheptyl. Heterocycloalkyl groups include, for example, 1-piperidinyl, 2-piperidinyl, 3-piperidinyl, 3-morpholinyl, 4-morpholinyl, tetrahydrofuran-2-yl, tetrahydrofuran-3-yl, tetrahydrothien-2-yl, tetrahydrothien-3-yl, 1-piperazinyl, 2-piperazinyl, and 1,4-diazabicyclooctane. Aryl groups include, for example, phenyl, indenyl, biphenyl, 1-naphthyl, 2-naphthyl, anthracenyl, and phenanthracenyl. Heteroaryl groups include, for example, 1-pyrrolyl, 2-pyrrolyl, 3-pyrrolyl, furyl, thienyl, imidazolyl, oxazolyl, thiazolyl, pyrazolyl, pyridyl, indolyl, quinolinyl, isoquinolinyl, benzoquinolinyl, carbazolyl, and diazaphenanthrenyl.

As used herein, halogen means the elements fluorine (F), chlorine (Cl), Bromine (Br), and iodine (I).

The subject platinum (IV) complexes can be prepared using standard chemical synthesis methods and materials known in the art.

Compounds of the subject invention also include physiologically-acceptable salts of the subject platinum complexes. Physiologically-acceptable salts includes salts of the platinum complexes of the invention which are prepared with acids or bases, depending on the particular substituents found on the subject complexes described herein. Examples of physiologically-acceptable base addition salts include sodium, potassium, calcium, ammonium, or magnesium salt. Examples of physiologically-acceptable acid addition salts include hydrochloric, hydrobromic, nitric, phosphoric, carbonic, sulphuric, and organic acids like acetic, propionic, benzoic, succinic, fumaric, mandelic, oxalic, citric, tartaric, maleic, and the like. Physiologically-acceptable salts of platinum complexes of the invention can be prepared using conventional techniques.

It will be appreciated by those skilled in the art that certain of the platinum complexes of the invention may contain one or more asymmetrically substituted carbon atoms which can give rise to stereoisomers. It is understood that the invention extends to all such stereoisomers, including enantiomers, and diastereoisomers and mixtures, including racemic mixtures thereof.

Specific examples of platinum complexes useful in the subject invention are shown below:

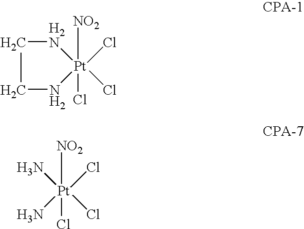

Methods of the invention comprise contacting a cell sample with a platinum complex and determining the level of uptake of the platinum complex into the cells. In one embodiment, a platinum complex of the invention is detected using an antibody that binds specifically to an epitope of the STAT protein. In another embodiment, a platinum complex of the invention is detected using an antibody that binds specifically to an immunogenic or antigenic determinant that has been conjugated to a platinum complex of the invention. Binding of the antibody to a platinum complex of the invention can be detected directly by using an antibody labeled directly or indirectly with a detectable label. In one embodiment, an antibody can be directly labeled by conjugating or coupling a detectable label, such as fluorescein, to the antibody. In a further embodiment, an antibody can be indirectly labeled by conjugating or coupling a moiety to the antibody that binds specifically to another moiety that comprises a detectable label. For example, an antibody can be conjugated with a biotin binding moiety such as avidin or streptavidin and then contacted with biotin that comprises a detectable label, such as fluorescein. Alternatively, the antibody can be detected using a second antibody that binds to the antibody bound to the platinum complex, wherein the second antibody is labeled directly or indirectly with a detectable label.

Antibodies contemplated within the scope of the invention include both polyclonal and monoclonal antibodies. Preferably, the antibody is a monoclonal antibody, or an antigen binding fragment thereof. Antigen binding fragments include, but are not limited to, F(ab′)₂, Fab′, Fab, and Fv, and can be prepared using standard methods known in the art. The antibody can be derived from any animal capable of producing antibodies to a platinum complex of the invention, or an immunogenic subunit thereof, and include, for example, primate, mouse, rat, goat, sheep, pig, and cow. Preferably, if the antibody is to be administered to humans, the antibody is a human antibody or is a “humanized” antibody derived from a non-human animal. Methods for humanizing non-human antibodies are known in the art and have been described in U.S. Pat. Nos. 5,530,101; 5,585,089; 5,693,762; 6,180,370; and 6,407,213. Antibodies of the invention can be prepared using standard techniques known in the art. In one embodiment, antibodies are prepared by immunizing an animal with a platinum complex of the invention, or an immunogenic subunit thereof. Monoclonal antibodies can be prepared using standard methods known in the art (Kohler et al., 1975).

In a further embodiment, a platinum complex of the invention can be detected using a polypeptide or a peptide that binds specifically to the platinum complex. Polypeptides and peptides that bind specifically to a particular platinum complex of the invention can be identified using standard methods in the art including, for example, screening of combinatorial libraries of peptides or phage display libraries. Methods and materials for preparing and screening combinatorial and phage display libraries are well known in the art (U.S. Pat. Nos. 5,432,018; 5,821,047; and 5,223,409). A peptide or polypeptide that binds specifically to a platinum complex of the invention can be detected by labeling directly or indirectly the polypeptide or peptide with a detectable label. Alternatively, a polypeptide or peptide bound to a platinum complex of the invention can be detected using an antibody that binds specifically to the polypeptide or peptide. The antibody can then be detected as described herein, e.g., by detecting a detectable label that is conjugated or otherwise bound to the antibody, or by using an antibody labeled with a detectable label.

In a still further embodiment, a platinum complex can be detected using a molecularly imprinted polymer (MIP) (Kriz et al., 1997) that has binding specificity for the platinum complex, or a portion thereof. MIPs are polymers that possess binding cavities with functional groups arranged in a complementary fashion to regions on a target analyte (Wu 2000; Byrne et al. 2002; Uezu et al. 1999). MIPs having binding specificity to a platinum complex useful in the subject invention can be prepared using standard methods and reagents known in the art (U.S. Pat. Nos. 5,821,311; 5,872,198; 5,959,050; 5,814,223; 5,630,978; and 5,916,445, and published U.S. Patent Application No. 20040072373). An MIP can be directly or indirectly labeled with a detectable label as described herein.

In one embodiment, the platinum complex itself is detectable by virtue of a substituent of the complex. For example, the complex designated herein as CPA51 can be detected by virtue of fluorescent emission from the luminol substituent where the complex is exposed to appropriate conditions. In a further embodiment, a detectable label may be coupled or conjugated either directly to a platinum complex of the invention, or indirectly, through an intermediate, such as, for example, a linker molecule. Linker molecules are known in the art. In another embodiment, a detectable label is directly coupled or conjugated to a binding moiety, such as an antibody, polypeptide, peptide or MIP, that binds to a platinum complex of the invention, or indirectly, though an intermediate (e.g., a linker molecule) using techniques known in the art. In one embodiment of the present invention, a detectable label can be directly, bound to the binding moiety that binds to a platinum complex of the invention. If the detectable label is to be directly bound, the label may comprise a functional group which is capable of binding to the binding moiety used with the invention. Alternatively, the detectable label may be indirectly bound, for example, using an avidin-biotin or streptavidin-biotin bridge wherein the avidin or biotin is labeled with a detectable label. In one embodiment, an antibody, polypeptide, peptide or MIP of the invention is conjugated with avidin and the detectable label is conjugated with biotin.

Detectable labels that can be used with the present invention include, but are not limited to, enzymes, radioisotopes, chemiluminescent and bioluminescent reagents, and fluorescent moieties. Enzymes that can be used include but are not limited to lucerifase, beta-galactosidase, acetylcholinesterase, horseradish peroxidase, glucose-6-phosphate dehydrogenase, and alkaline phosphatase. If the detectable label is an enzyme, then a suitable substrate that can be acted upon by the enzyme can be used for detection and measurement of enzyme activity. In one embodiment, if the detectable label is a peroxidase, the substrate can be hydrogen peroxide (H₂O₂) and 3-3′ diaminobenzidine or 4-chloro-1-naphthol and the like. Other substrates suitable for use with other enzymes are well known in the art. An example of a luminescent material includes luminol Examples of bioluminescent materials include, but are not limited to, luciferin, green fluorescent protein (GFP), enhanced GFP (Yang et al., 1996), and aequorin. Fluorescent moieties include, but are not limited to, umbelliferone, fluorescein, fluorescein isothiocyanate, Cascade Blue, rhodamine, dichlorotriazinylamine fluorescein, dansyl chloride, Texas Red, Oregon Green, cyanines (e.g., CY2, CY3, and CY5), allophycocyanine or phycoerytbrin. Isotopes that can be used include, but are not limited to, ¹²⁵I, ¹⁴C, ³⁵S, and ³H.

The subject invention also concerns methods for detection and quantification of STAT protein expression using a platinum complex of the present invention. In one embodiment, the STAT protein is STAT3. In one embodiment, a sample to be assayed for STAT protein is contacted with a platinum complex of the invention. Interaction of the STAT protein and the platinum complex is then detected. In one embodiment, the platinum complex is labeled with a detectable label. In another embodiment, the platinum complex is detected using a binding moiety, such as an antibody, polypeptide, peptide or MIP that binds specifically to the platinum complex. The subject invention can be used to monitor a person or animal for the onset, progression, or regression of a condition characterized by abnormal levels of STAT protein expression. Increased expression of a STAT protein relative to an earlier measurement or to a control measurement is indicative of onset or progression of a condition associated with abnormal STAT protein expression, such as an oncological, inflammatory, or neurological disorder. Methods of the invention include screening a patient who may have an oncological or inflammatory disorder. In one embodiment, cells to be tested are obtained from the patient and the level of STAT expressed in the cells is determined by contacting the cells with a platinum complex of the invention. The level of platinum complex associated with STAT proteins is then determined. The higher the levels of expression of a STAT protein, the higher the level of uptake of the platinum complex into the cell. The patient can be a human or other mammal, such as a primate (monkey, chimpanzee, ape, etc.), dog, cat, cow, pig, or horse, or other animals having an oncological disorder. Means for administering and formulating platinum complexes for administration to a patient are known in the art, examples of which are described herein. Oncological disorders include cancer and/or tumors of the bone, breast, kidney, mouth, larynx, esophagus, stomach, testis, cervix, head, neck, colon, ovary, lung, bladder, skin, liver, muscle, pancreas, prostate, blood cells (including lymphocytes), and brain. Inflammatory disorders include arthritis, multiple sclerosis, lupus, Crohn's disease, and related neurological and inflammatory connective tissue diseases (e.g., Sjögren's syndrome). Neurological disorders include Alzheimer's disease.

One embodiment of the invention concerns methods for diagnosis of an oncological disorder in a patient and for assessing aggressiveness (i.e., potential for metastasis) of the cancer or tumor of the disorder. The subject invention can be used to determine the level of a STAT protein expressed by a cancer or tumor cell of a patient. It is known that the more aggressive the cancer or tumor cell, the greater the level of expression of STAT proteins, such as STAT3. Thus, cancer or tumor cells of a patient can be screened using the materials and methods of the invention to determine the level of expression of a STAT protein associated with the cancer or tumor cells. An ordinarily skilled clinician can then determine, based upon the level of STAT expression observed, the aggressive potential of the cancer or tumor cells and can determine the most appropriate treatment protocol for the particular cancer or tumor. For example, a cancer or tumor cell that is determined to be highly aggressive (i.e., to have a high potential for metastasis in the patient's body) may suggest to the clinician to treat the patient with a more aggressive therapeutic protocol (e.g., radiation, surgery, chemotherapy, etc.) than, for example, a patient with a cancer or tumor that has been determined to have a relatively low aggressive potential.

In one embodiment, the method comprises contacting a cell with a platinum complex of the invention and detecting the platinum complex associated with a STAT protein. The cell can be a cell from a mammal, including human, monkey, chimpanzee, ape, dog, cat, horse, cow, or pig. Platinum complexes of the invention can be delivered to a cell either through direct contact with the cell or via a carrier means. Carrier means for delivering compositions to cells are known in the art and include, for example, encapsulating the platinum complex in a liposome moiety. Another means for delivery of a platinum complex of the invention to a cell comprises attaching the platinum complexes to a protein or nucleic acid that is targeted for delivery to the target cell. Published U.S. Patent Application Nos. 20030032594 and 20020120100 disclose amino acid sequences that can be coupled to another composition and that allows the composition to be translocated across biological membranes. Published U.S. Patent Application No. 20020035243 also describes compositions for transporting biological moieties across cell membranes for intracellular delivery.

The detection of a platinum complex of the invention within a patient body or tissue sample can be accomplished using standard techniques known in the art. For example, if the presence of a platinum complex is to be detected using histological means, a tissue or cell sample can be suitably prepared for contact with a platinum complex. The sample can then be suitably prepared and the presence of platinum complex detected using a binding moiety, such as an antibody, polypeptide, peptide or MIP, that can bind to the platinum complex as described herein. In one embodiment, the binding moiety comprises a detectable label suitable for use with histological techniques, e.g., an enzyme or a fluorescent label.

If the presence of a platinum complex is to be detected by imaging methods, such as by detection of radiation (scintigraphic imaging) or magnetic spin (magnetic resonance imaging), a detectable label can be used that comprises a radioisotope or a magnetic resonance (MR) enhancing agent. Magnetic resonance enhancing agents, such as Gadolinium (Gd) and Cobalt (Co), and the preparation thereof, have been described in U.S. Pat. Nos. 5,101,827; 5,059,415; and 6,534,039. In one embodiment, a moiety that binds to a platinum complex, such as an antibody, polypeptide, peptide or MIP, comprises a radiolabel or MR enhancing agent. Methods for preparing a platinum complex binding moiety that comprises a radioisotope or MR enhancing agent are known in the art (see, for example, U.S. Pat. Nos. 5,101,827; 5,059,415; 6,017,514; and 6,534,039). The imaging can be performed in vivo or in vitro, depending on the tissue or cells to be screened.

Detection and quantification of STAT protein in a sample can also be accomplished using flow cytometry. Flow cytometric methods and reagents for detection of an analyte in a sample are well known in the art.

In vivo application of the subject platinum complexes, and compositions containing them, can be accomplished by any suitable method and technique presently or prospectively known to those skilled in the art. The subject platinum complexes can be formulated in a physiologically- or pharmaceutically-acceptable form and administered by any suitable route known in the art including, for example, oral, nasal, rectal, and parenteral routes of administration. As used herein, the term parenteral includes subcutaneous, intradermal, intravenous, intramuscular, intraperitoneal, and intrasternal administration, such as by injection. Administration of the subject platinum complexes of the invention can be a single administration, or at continuous or distinct intervals as can be readily determined by a person skilled in the art.

The compounds of the subject invention can also be administered utilizing liposome technology, slow release capsules, implantable pumps, and biodegradable containers. These delivery methods can, advantageously, provide a uniform dosage over an extended period of time. The platinum complexes of the invention can also be administered in their salt derivative forms or crystalline forms.

Platinum complexes of the subject invention can be formulated according to known methods for preparing physiologically acceptable compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the platinum complex is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional physiologically-acceptable carriers and diluents which are known to those skilled in the art. Examples of carriers or diluents for use with the subject platinum complexes include ethanol, dimethyl sulfoxide, glycerol, alumina, starch, and equivalent carriers and diluents. To provide for the administration of such dosages for the desired therapeutic treatment, compositions of the invention will advantageously comprise between about 0.1% and 99%, and especially, 1 and 15% by weight of the total of one or more of the subject platinum complexes based on the weight of the total composition including carrier or diluent.

The subject invention also concerns a kit comprising in one or more containers at least one platinum complex useful in the subject invention. In one embodiment, the platinum complex is labeled with a detectable label. In another embodiment, where the platinum complex is provided in unlabeled form, the kit can optionally further comprise a detectable label that can be coupled, conjugated or otherwise bound to the platinum complex. In yet a further embodiment, the kit comprises an unlabeled platinum complex and a moiety that can bind to the platinum complex. In a specific embodiment, the binding moiety is an antibody, polypeptide, peptide, or molecularly imprinted polymer that is capable of binding to the platinum complex. Optionally, the binding moiety can be provided with a detectable label already bound to the moiety, or if the binding moiety is provided in unlabeled form, the kit can comprise a detectably labeled moiety that can bind to the unlabeled binding moiety or the kit can comprise a detectable label that can coupled, conjugated or otherwise bound to the unlabeled binding moiety.

TABLE 1

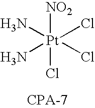

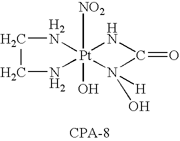

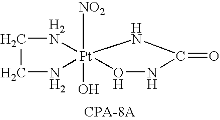

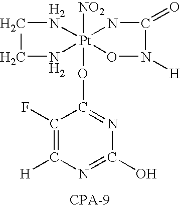

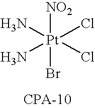

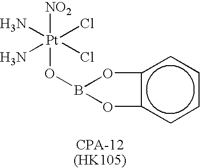

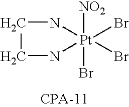

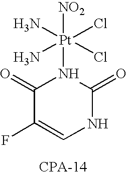

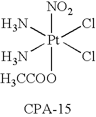

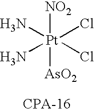

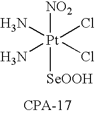

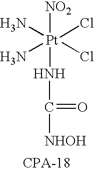

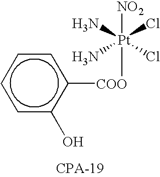

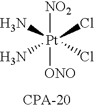

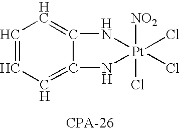

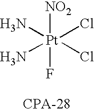

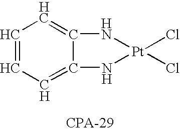

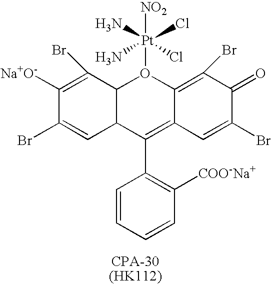

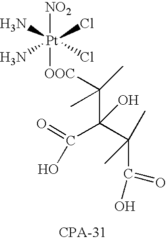

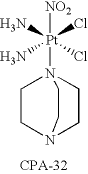

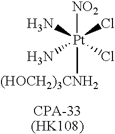

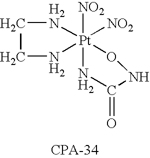

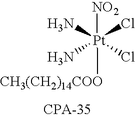

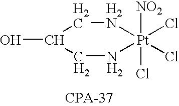

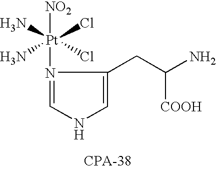

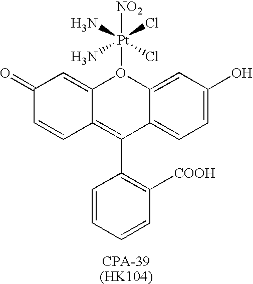

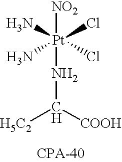

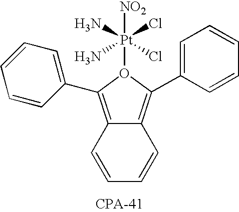

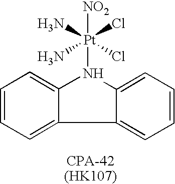

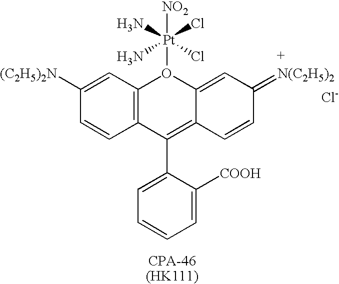

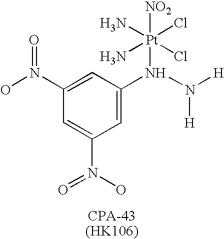

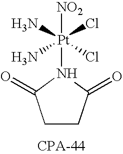

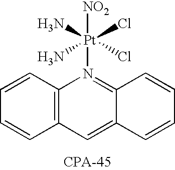

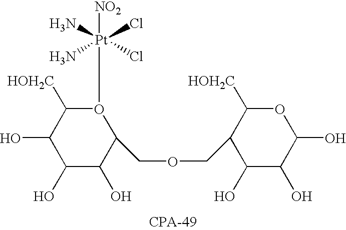

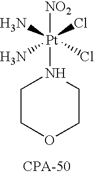

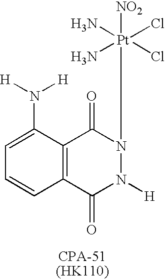

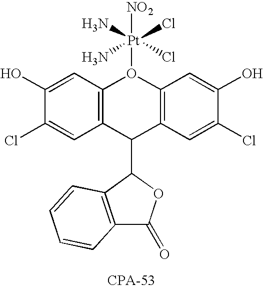

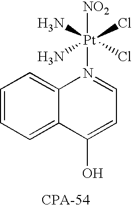

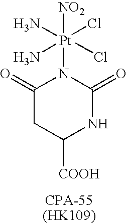

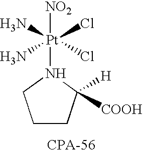

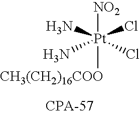

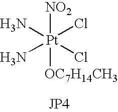

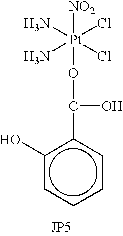

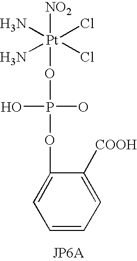

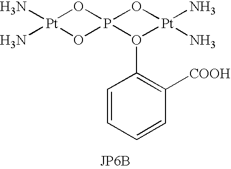

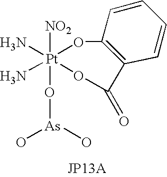

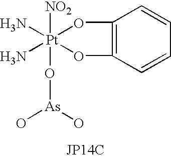

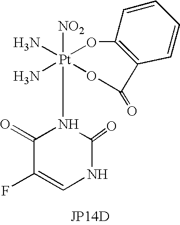

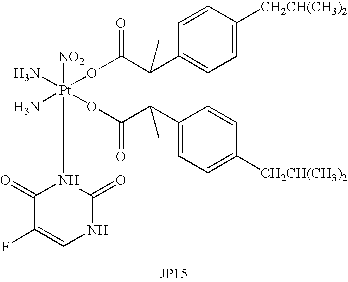

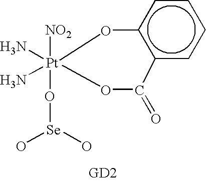

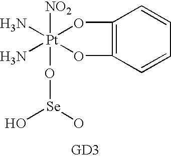

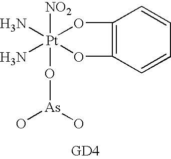

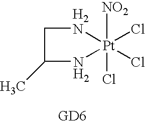

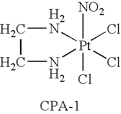

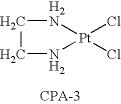

All patents, patent applications, provisional applications, and publications referred to or cited herein are incorporated by reference in their entirety, including all figures and tables, to the extent they are not inconsistent with the explicit teachings of this specification.

### Materials and Methods

Following are examples which illustrate procedures for practicing the invention. These examples should not be construed as limiting. All percentages are by weight and all solvent mixture proportions are by volume unless otherwise noted.

### Example 1

Two syngeneic variants of murine pancreatic cell lines were compared. The H7 cell line expresses high levels of STAT3 while the H2 line has been genetically transformed to express low levels of STAT3. Cell lines were maintained in DMEM with 10% FBS, L-glutamine, and 100 u/mL pen-strep. These were maintained in culture flasks incubated at 37° C. and 5% CO₂.

### Fluorescence Microscopy

Approximately 10⁶ cells were placed in individual wells of 24 well plates and treated at 25 μM concentration of various platinum complexes. Stock platinum complexes of 250 uM concentrations were prepared in 20% DMSO with standard media. 100 uL of this stock was added to each well together with 900 uL of counted cells in media. After 36 hours of treatment, contents of each well were removed and cells were gently washed five times with PBS. Cells were then scraped from the growth surface or collected by treatment with trypsin and wet-mounted in PBS on a glass slide with glass cover slip. Cells were then viewed on a Leica Inverted Fluoroscope and photographs were taken with an attached 35 mm Nikon camera with 400 ISO film. Excitation wavelengths were estimated for each compound, based upon their ligands, and the appropriate wavelength range was selected for each complex before viewing.

TABLE 2

| | | | | | Results shown |
|---|---|---|---|---|---|
| Cell line* | Drug | Mag | Upper | Lower | in Figure |
| Panc H2 | CPA51 = HK110 | 640x | Fluor | Light | 1A and 1B |
| | (Luminol) | | | | |
| Panc H2 | CPA51 = HK110 | 400x | Light | Fluor | 2A and 2B |
| | (Luminol) | | | | |
| Panc H2 | CPA51 = HK110 | 400x | Light | Fluor | 3A and 3B |
| | (Luminol) | | | | |
| Panc H7 | CPA51 = HK110 | 400x | Light | Fluor | 4A and 4B |
| | (Luminol) | | | | |
| Panc H7 | CPA51 = HK110 | 400x | Light | Fluor | 5A and 5B |
| | (Luminol) | | | | |
| Panc H7 | CPA51 = HK110 | 400x | Light | Fluor | 6A and 6B |
| | (Luminol) | | | | |
| Panc H7 | CPA51 = HK110 | 400x | Light | Fluor | 7A and 7B |
| | (Luminol) | | | | |

H2 expresses low levels of STAT3 whereas H7 expresses high levels

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 5,530,101
U.S. Pat. No. 5,585,089
U.S. Pat. No. 5,693,762
U.S. Pat. No. 6,180,370
U.S. Pat. No. 6,407,213
U.S. Pat. No. 5,432,018
U.S. Pat. No. 5,821,047
U.S. Pat. No. 5,223,409
U.S. Pat. No. 5,101,827
U.S. Pat. No. 5,059,415
U.S. Pat. No. 6,534,039
U.S. Pat. No. 5,101,827
U.S. Pat. No. 6,017,514
U.S. Pat. No. 5,821,311
U.S. Pat. No. 5,872,198
U.S. Pat. No. 5,959,050
U.S. Pat. No. 5,814,223
U.S. Pat. No. 5,630,978
U.S. Pat. No. 5,916,445
Published U.S. Patent Application No. 20040072373
Published U.S. Patent Application No. 20030032594
Published U.S. Patent Application No. 20020120100
Published U.S. Patent Application No. 20020035243
Akira, S. (2000) “Roles of STAT3 defined by tissue-specific gene targeting” Oncogene 19:2607-2611.
Ardizzoni, A., Antonelli, G., Grossi, F., Tixi, L., Cafferata, M., Rosso, R. (1999) “The combination of etoposide and cisplatin in non-small-cell lung cancer (NSCLC)” Ann. Oncol. 10:S13-17.
Bowman, T., Garcia, R., Turkson, J., Jove, R. (2000a) “STATs in oncogenesis” Oncogene 19:2474-2488.
Bromberg, J. F., Horvath, C. M., Wen, Z., Schreiber, R. D., Darnell, J. E., Jr. (1996) “Transcriptionally active Stat1 is required for the antiproliferative effects of both interferon alpha and interferon gamma” Proc. Natl. Acad. Sci. USA 93:7673-7678.
Bromberg, J. F., Horvath, C. M., Besser, D., Lathem, W. W., Darnell, J. E., Jr. (1998) “Stat3 activation is required for cellular transformation by v-src” Mol. Cell. Biol. 18:2553-2558.
Bromberg, J. F., Wrzeszczynska, M. H., Devgan, G., Zhao, Y., Pestell, R. G., Albanese, C., Darnell, J. E., Jr. (1999) “Stat3 as an oncogene” Cell 98:295-303.
Byrne, M. E., Park, K., Pappas, N. A. (2002) “Molecular imprinting within hydrogels” Advanced Drug Delivery Reviews 54:149-161.
Catlett-Falcone, R., Landowski, T. H., Oshiro, M. M., Turkson, J., Levitzki, A., Savino, R., Ciliberto, G., Moscinski, L., Fernandez-Luna, J. L., Nunez, G., Dalton, W. S., Jove, R. (1999a) “Constitutive activation of Stat3 signaling confers resistance to apoptosis in human U266 myeloma cells” Immunity 10:105-115.
Catlett-Falcone, R., Dalton, W. S., Jove, R. (1999b) “STAT proteins as novel targets for cancer therapy. Signal transducer an activator of transcription” Curr. Opin. Oncol. 11:490-496.
Coffer, P. J., Koenderman, L., de Groot, R. P. (2000) “The role of STATs in myeloid differentiation and leukemia” Oncogene 19:2511-2522.
Darnell, J. E., Jr., Kerr, I. M., Stark, G. R. (1994) “Jak-STAT pathways and transcriptional activation in response to IFNs and other extracellular signaling proteins” Science 264:1415-1421.
Darnell, J. E., Jr. (1997) “STATs and Gene Regulation” Science 277:1630-1635.
Epling-Burnette, P. K., Lui, J. H., Catlette-Falcone, R., Turkson, J., Oshiro, M., Kothapalli, R., Li, Y., Wang, J.-M., Yang-Yen, H.-F., Karras, J., Jove, R., Loughran, T. P., Jr. (2001) “Inhibition of STAT3 signaling leads to apoptosis of leukemic large granular lymphocytes and decreased Mcl-1 expression” J. Clin. Invest 107:351-362.
Fukada, T., Hibi, M., Yamanaka, Y., Takahashi-Tezuka, M., Fujitani, Y., Yamaguchi, T., Nakajima, K., Hirano, T. (1996) “Two signals are necessary for cell proliferation induced by a cytokine receptor gp130: involvement of STAT3 in anti-apoptosis” Immunity 5:449-460.
Garcia, R., Jove, R. (1998) “Activation of STAT transcription factors in oncogenic tyrosine kinase signaling” J. Biomed. Sci. 5:79-85.
Garcia, R., Bowman, T. L., Niu, G., Yu, H., Minton, S., Muro-Cacho, C. A., Cox, C. E., Falcone, R., Fairclough, R., Parson, S., Laudano, A., Gazit, A., Levitzki, A., Kraker, A., Jove, R. (2001) “Constitutive activation of Stat3 by the Src and JAK tyrosine kinases participates in growth regulation of human breast carcinoma cells” Oncogene 20:2499-2513.
Grandis, J. R., Drenning, S. D., Chakraborty, A., Zhou, M. Y., Zeng, Q., Pitt, A. S., Tweardy, D. J. (1998) “Requirement of Stat3 but not Stat1 activation for epidermal growth factor receptor-mediated cell growth In vitro” J. Clin. Invest. 102:1385-1392.
Grandis, J. R., Drenning, S. D., Zeng, Q., Watkins, S. C., Melhem, M. F., Endo, S., Johnson, D. E., Huang, L., He, Y., Kim, J. D. (2000a) “Constitutive activation of Stat3 signaling abrogates apoptosis in squamous cell carcinogenesis in vivo” Proc. Natl. Acad. Sci. USA 97:4227-4232.
Hirano, T., Ishihara, K., Hibi, M. (2000) “Roles of STAT3 in mediating the cell growth, differentiation and survival signals relayed through the IL-6 family of cytokine receptors” Oncogene 19:2548-2556.
Irish, J. M., Hovland, R., Krutzik, P. O., Perez, O. D., Bruserud, Ø., Gjertsen, B. T., Nolan, G. P., (2004) “Single Cell Profiling of Potentiated Phospho-Protein Networks in Cancer Cells” Cell 118:217-228.
Kohler, G., C. Milstein (1975) “Continuous cultures of fused cells secreting antibody of predefined specificity” Nature 256(5517):495-497.
Kotenko, S. V., Pestka, S. (2000) “Jak-Stat signal transduction pathway through the eyes of cytokine class II receptor complexes” Oncogene 19:2557-2565.
Kriz, D., O. Ramstrom, K. Mosbach (1997) “Molecular imprinting-based biomimetic sensors could provide an alternative to often unstable biosensors for industry, medicine, and environmental analysis” Analytical Chemistry 69:345A-349A.
Krutzik, P. O., Irish, J. M., Nolan, G. P., Perez, O. D. (2004) “Analysis of Protein Phosphorylation and Cellular Signaling Events by Flow Cytometry: Techniques and Clinical Applications” Clinical Immunology 110:206-221.
Lin, T. S., Mahajan, S., Frank, D. A. (2000) “STAT signaling in the pathogenesis and treatment of leukemias” Oncogene 19:2496-2504.
Nielsen, M., Kaltoft, K., Nordahl, M., Ropke, C., Geisler, C., Mustelin, T., Dobson, P., Svejgaard, A., Odum, N. (1997) “Constitutive activation of a slowly migrating isoform of Stat3 in mycosis fungoides: tyrphostin AG490 inhibits Stat3 activation and growth of mycosis fungoides tumor cell lines” Proc. Natl. Acad. Sci. USA 94:6764-6769.
Nielsen, M., Kaestel, C. G., Eriksen, K. W., Woetmann, A., Stokkedal, T., Kaltoft, K., Geisler, C., Ropke, C., Odum, N. (1999) “inhibition of constitutively activated Stat3 correlates with altered Bcl-2fBax expression and induction of apoptosis in mycosis fungoides tumor cells” Leukemia 13:735-738.
Nitiss, J. L. (2002) “A copper connection to the uptake of platinum anticancer drugs” Proc. Natl. Acad. Sci. USA 99:13963-13965.
Persons, D. L., Yazlovitskaya, E. M., Cui, W., Felling, J. C. (1999) “Cisplatin-induced Activation of Mitogen-activated Protein Kinases in Ovarian Carcinoma Cells: Inhibition of Extracellular Signal-regulated Kinase Activity Increases Sensitivity to Cisplatin” Clin. Cancer Res. 5:1007-1014.
Sanchez-Perez, I., Murguia, J. R., Perona, R. (1998) “Cisplatin induces a persistent activation of JNK that is related to cell death” Oncogene 16:533-540.
Schindler, C., Darnell, J. E., Jr. (1995) “Transcriptional responses to polypeptide ligands: the JAK-STAT pathway” Annu. Rev. Biochem. 64:621-651.
Smithgall, T. E., Briggs, S. D., Schreiner, S., Lerner, E. C., Cheng, H., Wilson, M. B. (2000) “Control of myeloid differentiation and survival by Stats” Oncogene 19:2612-2618.
Song, J. I., Grandis, J. R. (2000) “STAT signaling in head and neck cancer” Oncogene 19:2489-2495.
Stark, G. R., Kerr, I. M., Williams, B. R., Silverman, R. H., Schreiber, R. D. (1998) “How cells respond to interferons” Annu. Rev. Biochem. 67:227-264.
Turkson, J., Bowman, T., Garcia, R., Caldenhoven, E., de Groot, R. P., Jove, R. (1998) “Stat3 activation by Src induces specific gene regulation and is required for cell transformation” Mol. Cell. Biol. 18:2545-2552.
Turkson, J., Jove, R. (2000) “STAT proteins: novel molecular targets for cancer drug discovery” Oncogene 19:6613-6626.
Turkson, J., Zhang, S. Palmer, J. Kay, H. Mora, L., Stanko, J., Jove, R. (2004) “Inhibition of constitutive Stat3 activation by novel platinum complexes with potent anti-tumor activity” Molecular Cancer Therapeutics 3:533-1542.
Uezu, K., Yoshida, M., Goto, M., Furusaki, S. (1999) “Molecular recognition using surface template polymerization” American Chemical Society 29:12-18.
Wu, C. (2000) “Molecules Leave Their Mark” Science News 157:186-188.
Yang, T. T. et al. (1996) “Optimized Codon Usage and Chromophore Mutations Provide Enhanced Sensitivity with the Green Fluorescent Protein” Nucleic Acid Research 24(22):4592-4593.

1-120. (canceled)

121. A platinum complex having the structure shown in formula IA or IB: 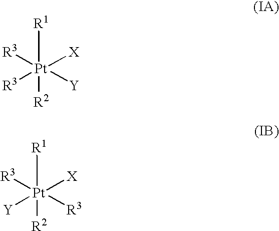
wherein
X and Y are, independently, any halogen, —NO₂, —ONO, or the structure: 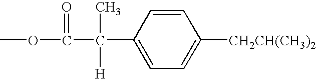
or X and Y together form the structure: 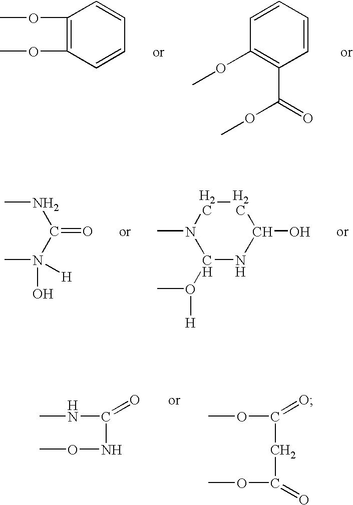
R¹ is —NO₂, —ONO, Cl, Br or F;
R² is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 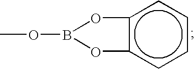
any of which can be substituted with any halogen, —NH₂, —COOH, —OH, alkoxy, cycloalkoxy;
R³ is, independently, —NH₃, —NHR⁷, —NH₂R⁷, —NH(R⁷)₂, or —N(R⁷)₃;
R⁷ is H, C₁₋₆ alkyl, alkoxy, or aryl, any of which can be optionally substituted with any halogen, —NO₂, or —COOH;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
or a pharmaceutically acceptable salt thereof.

122. The platinum complex according to claim 1, wherein X and Y are, independently, selected from the group consisting of F, Cl, Br, and I.

123. The platinum complex according to claim 1, wherein X and Y are both Cl.

124. The platinum complex according to claim 1, wherein R¹ is —NO₂.

125. The platinum complex according to claim 1, wherein R³ is —NH₃.

126. A platinum complex having the structure shown in formula II: 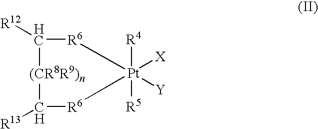
wherein
X and Y are, independently, any halogen, or the structure: 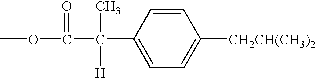
or X and Y together form the structure: 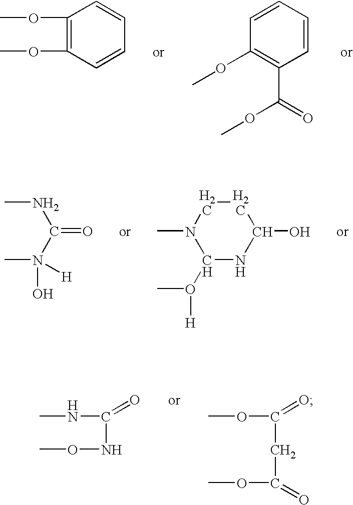
R⁴ is —NO₂ or —ONO;
R⁵ is any halogen, —OH, —ONO, —ONO₂, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 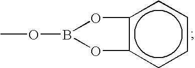
any of which can be substituted with any halogen, —NH₂, —COOH, —OH, or Y and R⁵ form the structure: 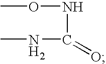
or X and Y together form the structure: 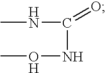
R⁶ is, independently, NH₂, NH, NHR⁷, N(R⁷)₂, NHR⁸, N(R⁸)₂, NHR⁹, N(R⁹)₂, or NR⁸R⁹;
R⁷ is H, C₁₋₆ alkyl, alkoxy, aryl, any of which can be optionally substituted with any halogen, —NO₂, or —COOH;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H or C₁₋₆ alkyl, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof.

127. The platinum complex according to claim 126, wherein X and Y are, independently, selected from the group consisting of F, Cl, Br, and I.

128. The platinum complex according to claim 126, wherein X and Y are both Cl.

129. The platinum complex according to claim 126, wherein R⁴ is —NO₂.

130. The platinum complex according to claim 126, wherein R⁶ is —NH₂.

131. A platinum complex having the structure shown in formula III or formula IVA or IVB: 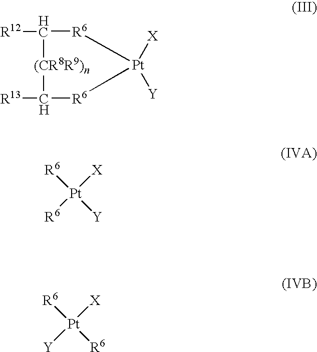
wherein
X and Y are, independently, any halogen, —NO₂, —ONO, or X and Y together form the structure: 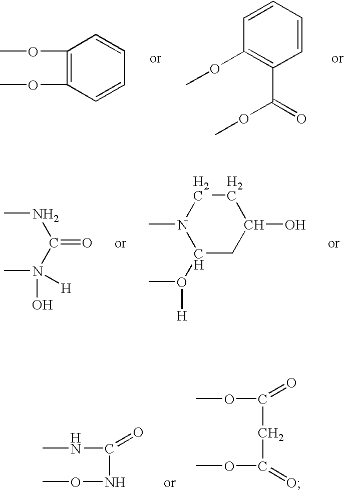
R⁶ is, independently, Cl, Br, F, NO₂, ONO, NHR⁸, NH₂, NHR¹², NR¹², N(R¹²)₂, NHR¹³, NR¹³, N(R¹³)₂, or NR¹²R¹³;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H, C₁₋₆ alkyl, or —OH, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof.

132. The platinum complex according to claim 131, wherein X and Y are, independently, selected from the group consisting of F, Cl, Br, and I.

133. The platinum complex according to claim 131, wherein X and Y are both Cl.

134. A platinum complex having the structure shown in formula VA or VB or formula VI: 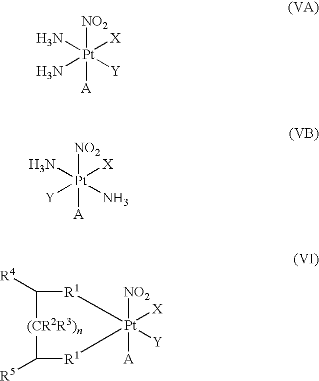
wherein
X and Y are, independently, any halogen, —OH, H₂O, or —SO(CH₃)₂;
or X and Y together form the structure: 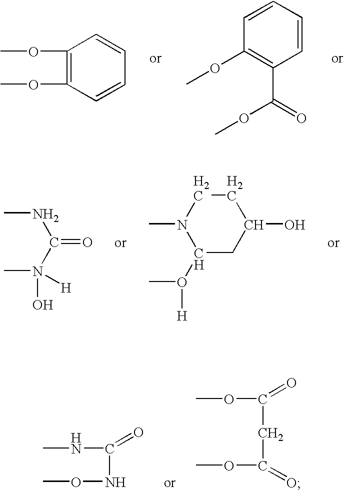
and A can be any of the following: 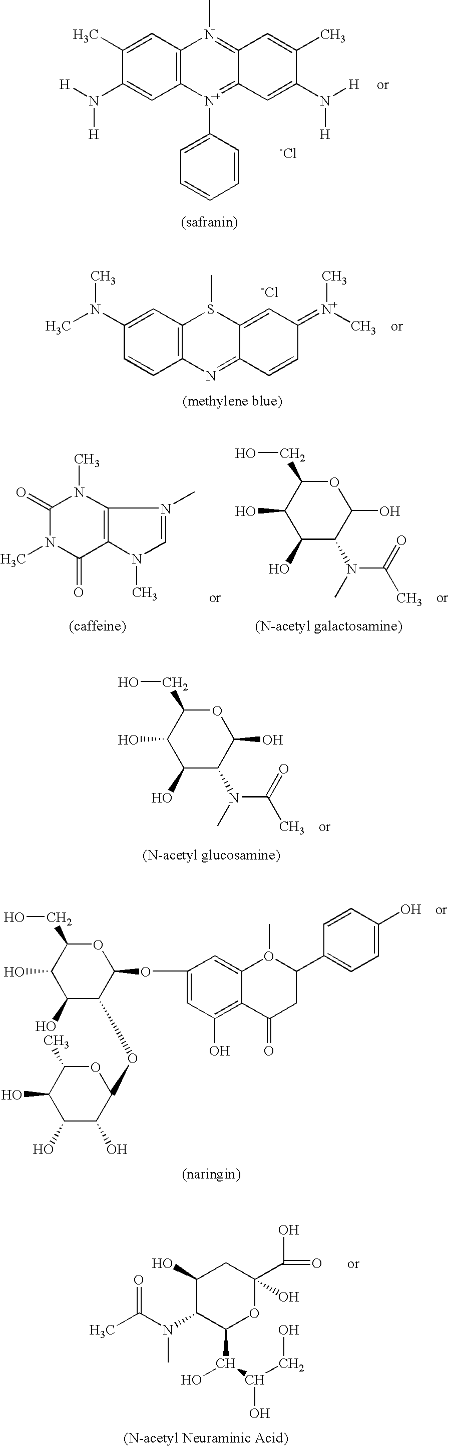 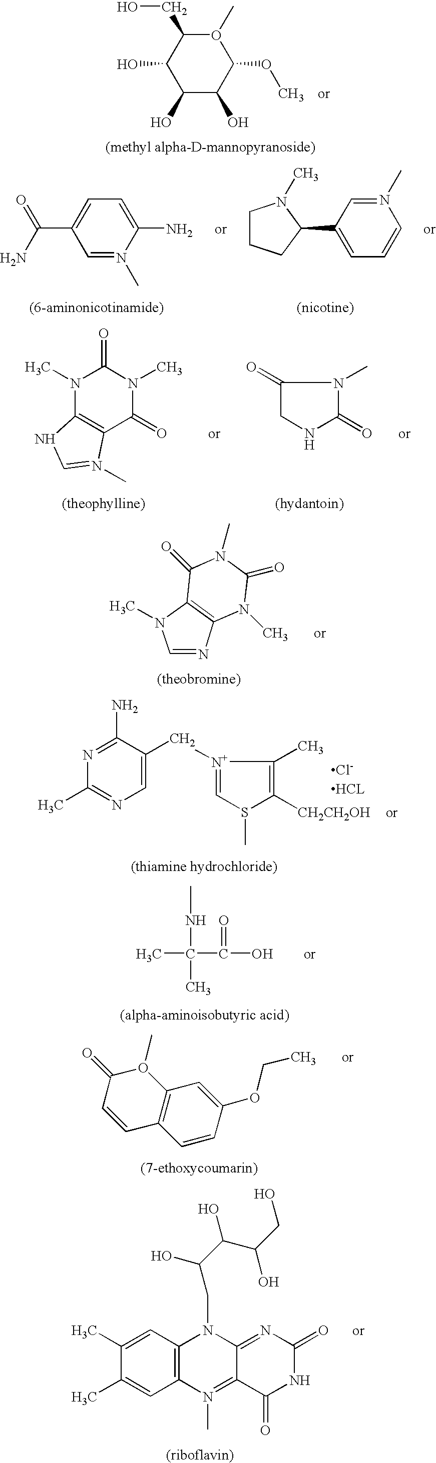 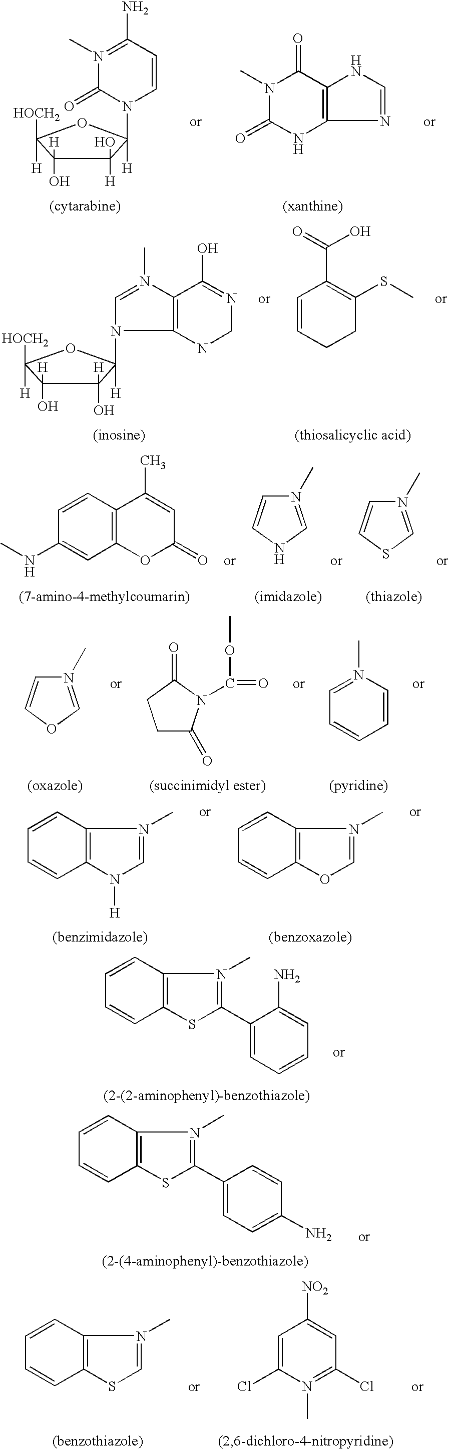 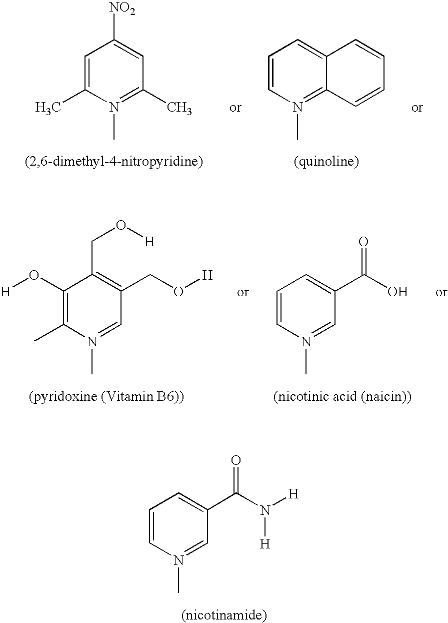
and wherein
R¹ is, independently, NH₂, NH, NR⁴, NHR⁴, N(R⁴)₂, NR⁵, NHR⁵, N(R⁵)₂, or NR⁴R⁵;
R² and R³ are, independently, H, —OH, C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
R⁴ and R⁵ independently, independently, H or C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl or R⁴ and R⁵ together form a cycloalkyl, cycloalkoxy, aryl, aryloxy, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
n is any integer from 0 to 6;
or a pharmaceutically acceptable salt thereof.

135. The platinum complex according to claim 134, wherein X and Y are, independently, selected from the group consisting of F, Cl, Br, and I.

136. The platinum complex according to claim 134, wherein X and Y are both Cl.

137. A method for screening for the level of expression of a STAT protein in a cell, said method comprising contacting a cell with a composition comprising:
a) a platinum complex having the structure shown in formula IA or IB: 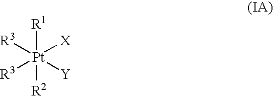
wherein
X and Y are, independently, any halogen, —NO₂, —ONO, or the structure: 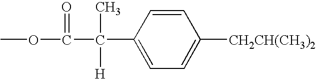
or X and Y together form the structure: 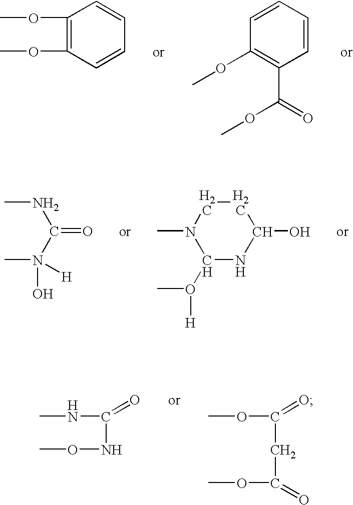
R¹ is —NO₂, —ONO, Cl, Br or F;
R² is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 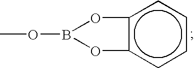
any of which can be substituted with any halogen, —NH₂, —COOH, —OH, alkoxy, cycloalkoxy;
R³ is, independently, —NH₃, —NHR⁷, —NH₂R⁷, —NH(R⁷)₂, or —N(R⁷)₃;
R⁷ is H, C₁₋₆ alkyl, alkoxy, or aryl, any of which can be optionally substituted with any halogen, —NO₂, or —COOH;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
or a pharmaceutically acceptable salt thereof; or
b) a platinum complex having the structure shown in formula II: 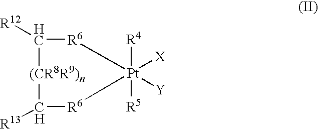
wherein
X and Y are, independently, any halogen, or the structure: 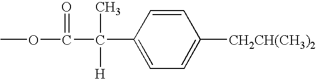
or X and Y together form the structure: 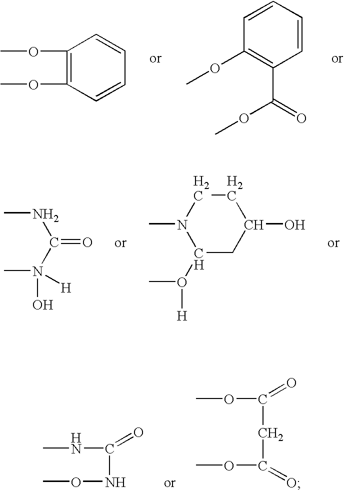
R⁴ is —NO₂ or —ONO;
R⁵ is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 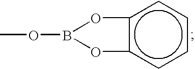
any of which can be substituted with any halogen, —NH₂, —COOH, —OH, or Y and R⁵ form the structure: 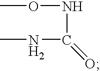
or X and Y together form the structure: 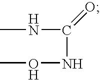
R⁶ is, independently, NH₂, NH, NHR⁷, N(R⁷)₂, NHR⁸, N(R⁸)₂, NHR⁹, N(R⁹)₂, or NR⁸R⁹;
R⁷ is H, C₁₋₆ alkyl, alkoxy, aryl, any of which can be optionally substituted with any halogen, —NO₂, or —COOH;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H or C₁₋₆ alkyl, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof; or
c) a platinum complex having the structure shown in formula III or formula IVA or IVB: 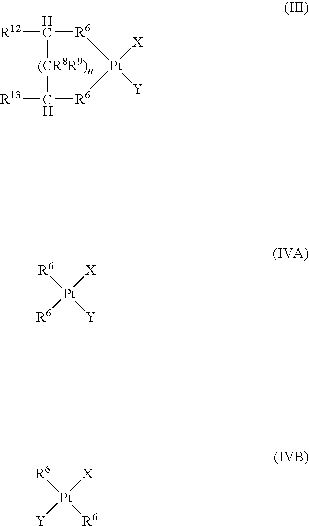
wherein
X and Y are, independently, any halogen, —NO₂, —ONO, or X and Y together form the structure: 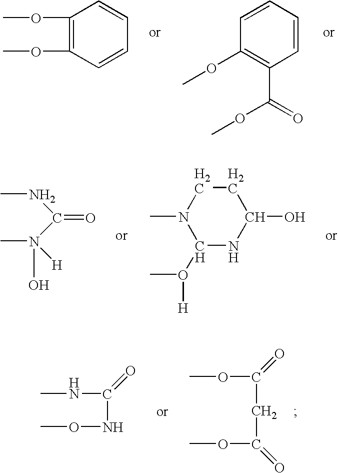
R⁶ is, independently, Cl, Br, F, NO₂, ONO, NHR⁸, NH₂, NHR¹², NR¹², N(R¹²)₂, NHR¹³, NR¹³, N(R¹³)₂, or NR¹²R¹³;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H, C₁₋₆ alkyl, or —OH, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof; or
d) a platinum complex having the structure shown in formula VA or VB or formula VI: 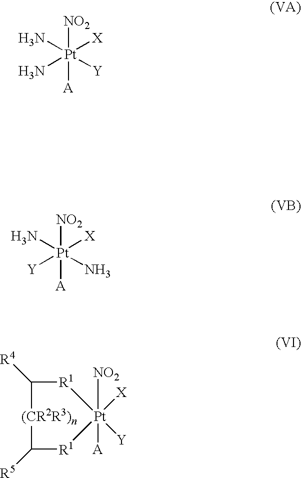
wherein
X and Y are, independently, any halogen, —OH, H₂O, or —SO(CH₃)₂;
or X and Y together form the structure: 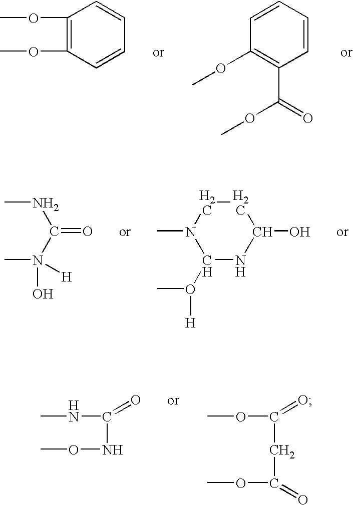
and A can be any of the following: 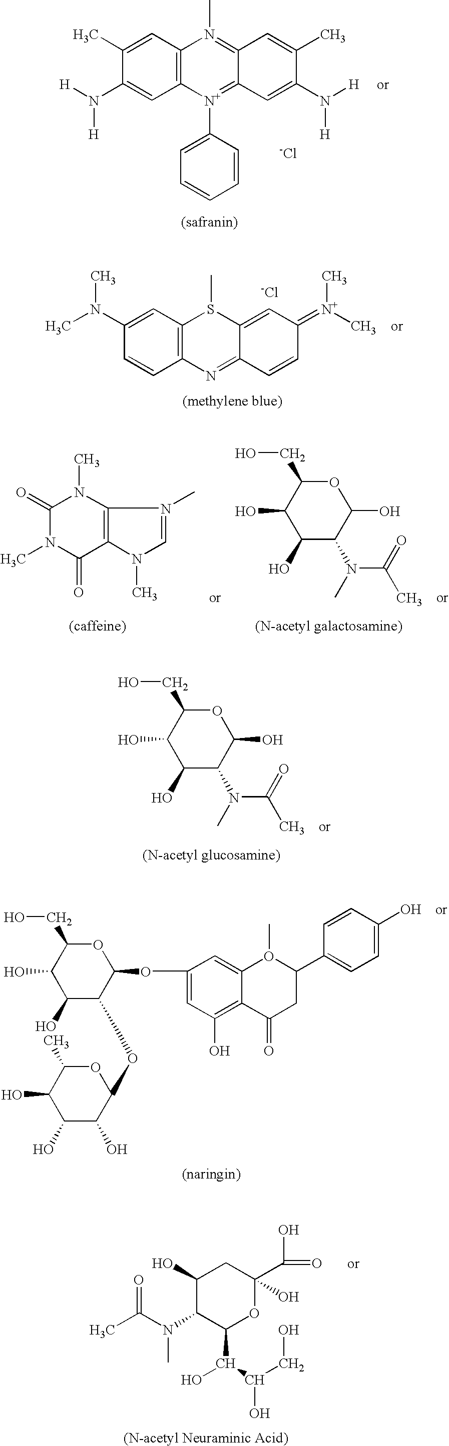 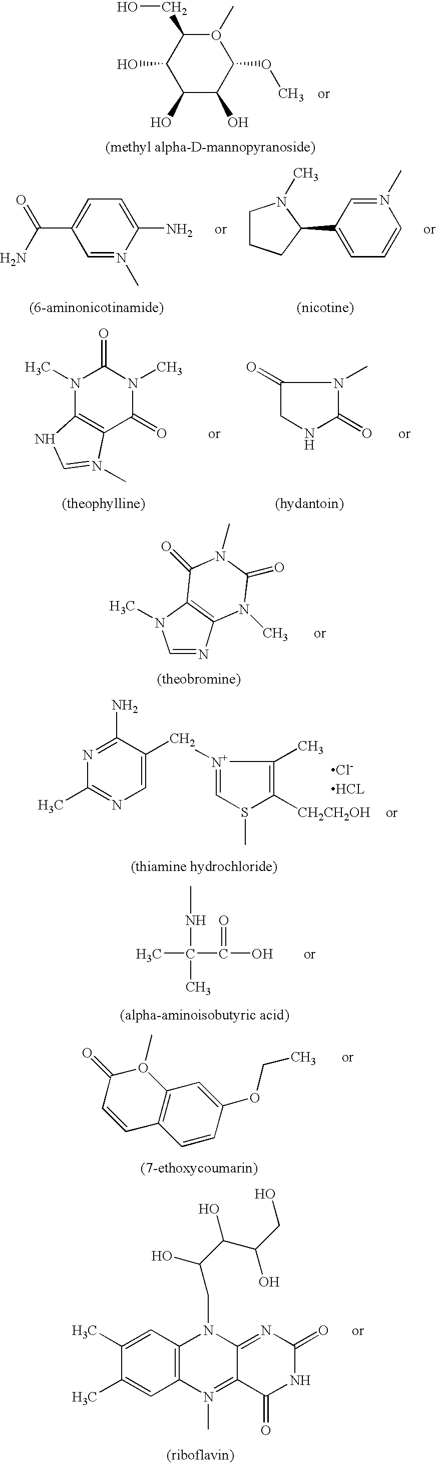 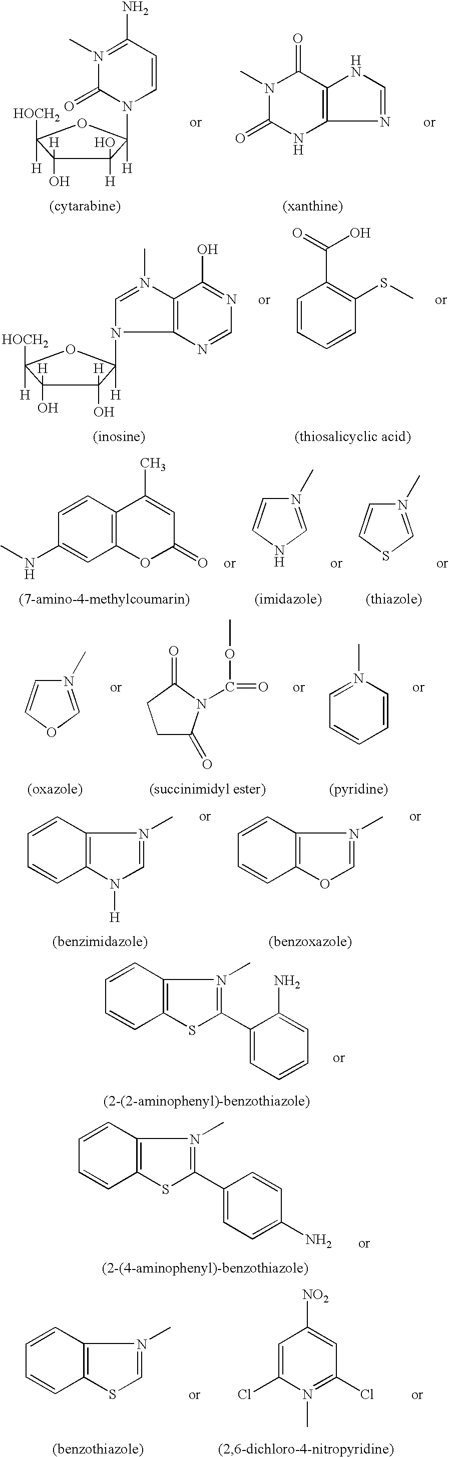 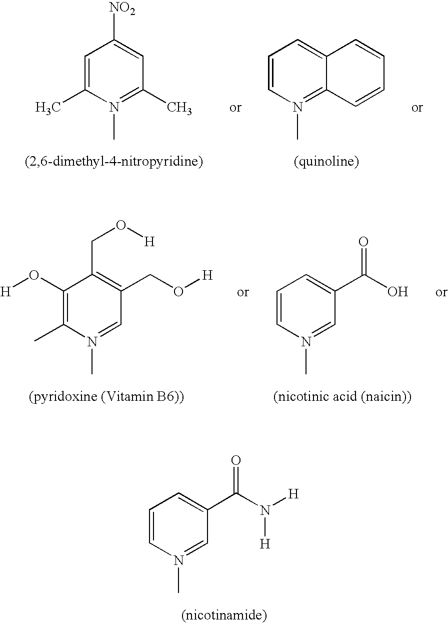
and wherein
R¹ is, independently, NH₂, NH, NR⁴, NHR⁴, N(R⁴)₂, NR⁵, NHR⁵, N(R⁵)₂, or NR⁴R⁵;
R² and R³ are, independently, H, —OH, C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
R⁴ and R⁵ are, independently, H or C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl or R⁴ and R⁵ together form a cycloalkyl, cycloalkoxy, aryl, aryloxy, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
n is any integer from 0 to 6;
or a pharmaceutically acceptable salt thereof; and
determining the level of said platinum complex in said cell or determining the level of uptake of said platinum complex into said cell, and correlating the level of said platinum complex or the level of uptake of said platinum complex with the level of said STAT protein in said cell.

138. The method according to claim 137, wherein the level of said platinum complex in said cell or the level of uptake of said platinum complex is detected using:
i) an antibody, or an antigen binding fragment thereof, that binds specifically to an epitope of said STAT protein; or
ii) an antibody, or an antigen binding fragment thereof, that binds specifically to an immunogenic or antigenic determinant of or conjugated to said platinum complex.

139. The method according to claim 138, wherein said antibody is directly or indirectly labeled with a detectable label and the presence of said label in said cell is qualitatively or quantitatively detected.

140. The method according to claim 138, wherein said antibody can be detected using a second antibody that binds to said antibody, wherein said second antibody is directly or indirectly labeled with a detectable label and the presence of said label in said cell is qualitatively or quantitatively detected.

141. The method according to claim 137, wherein the level of said platinum complex in said cell or the level of uptake of said platinum complex into said cell is detected using a polypeptide or a peptide that binds specifically to said platinum complex.

142. The method according to claim 141, wherein said polypeptide or peptide that binds specifically to said platinum complex is directly or indirectly labeled with a detectable label and the presence of said label in said cell is qualitatively or quantitatively detected.

143. The method according to claim 141, wherein said peptide or polypeptide that binds specifically to said platinum complex is detected using an antibody, optionally directly or indirectly labeled with a detectable label, that binds specifically to said polypeptide or peptide.

144. The method according to claim 137, wherein the level of said platinum complex in said cell or the level of uptake of said platinum complex into said cell is detected by detection of a substituent or ligand of said platinum complex.

145. The method according to claim 137, wherein the level of said platinum complex in said cell or the level of uptake of said platinum complex into said cell is detected by detecting a detectable label that is coupled or conjugated directly or indirectly to said platinum complex and the presence of said label in said cell is qualitatively or quantitatively detected.

146. A kit comprising in one or more containers a platinum complex
a) having the structure shown in formula IA or IB: 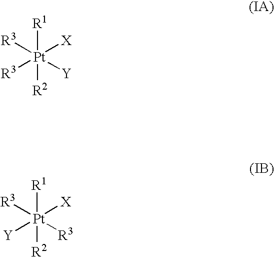
wherein
X and Y are, independently, any halogen, —NO₂, —ONO, or the structure: 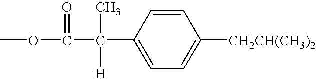
or X and Y together form the structure: 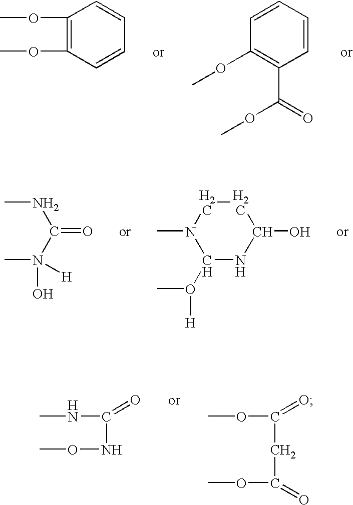
R¹ is —NO₂, —ONO, Cl, Br or F;
R² is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹, —NHR¹⁰R¹¹, OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 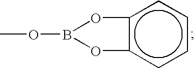
any of which can be substituted with any halogen, —NH₂, —COOH, —OH, alkoxy, cycloalkoxy;
R³ is, independently, —NH₃, —NHR⁷, —NH₂R⁷, —NH(R⁷)₂, or —N(R⁷)₃;
R⁷ is H, C₁₋₆ alkyl, alkoxy, or aryl, any of which can be optionally substituted with any halogen, —NO₂, or —COOH;
R¹⁰ and R¹¹ are, independently, H, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
or a pharmaceutically acceptable salt thereof; or
b) a platinum complex having the structure shown in formula II: 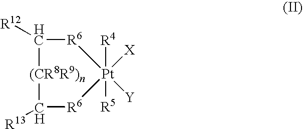
wherein
X and Y are, independently, any halogen, or the structure: 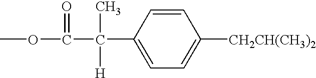
or X and Y together form the structure: 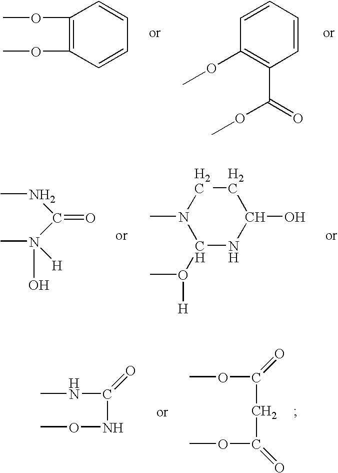
R⁴ is —NO₂ or —ONO;
R⁵ is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 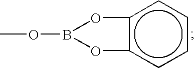
any of which can be substituted with any halogen, —NH₂, —COOH, —OH, or Y and R⁵ form the structure: 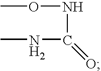
or X and Y together form the structure: 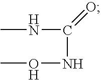
R⁶ is, independently, NH₂, NH, NHR⁷, N(R⁷)₂, NHR⁸, N(R⁸)₂, NHR⁹, N(R⁹)₂, or NR⁸R⁹;
R⁷ is H, C₁₋₆ alkyl, alkoxy, aryl, any of which can be optionally substituted with any halogen, —NO₂, or —COOH;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H or C₁₋₆ alkyl, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof; or
c) a platinum complex having the structure shown in formula III or formula IVA or IVB: 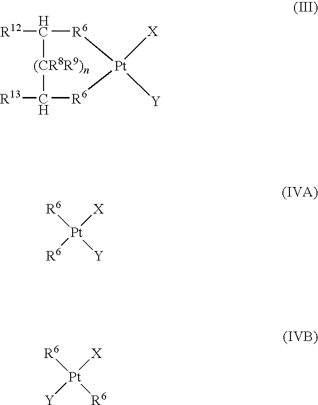
wherein
X and Y are, independently, any halogen, —NO₂, —ONO, or X and Y together form the structure: 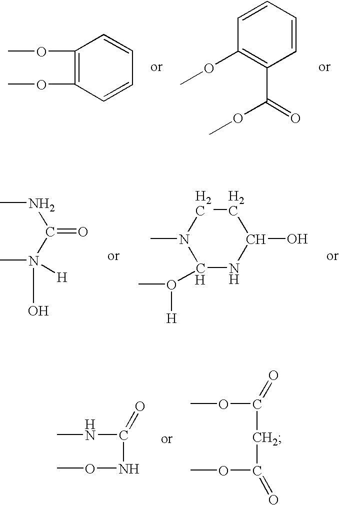
R⁶ is, independently, Cl, Br, F, NO₂, ONO, NHR⁸, NH₂, NHR¹², NR¹², N(R¹²)₂, NHR¹³, NR¹³, N(R¹³)₂, or NR¹²R¹³;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H, C₁₋₆ alkyl, or —OH, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof; or
d) a platinum complex having the structure shown in formula VA or VB or formula VI: 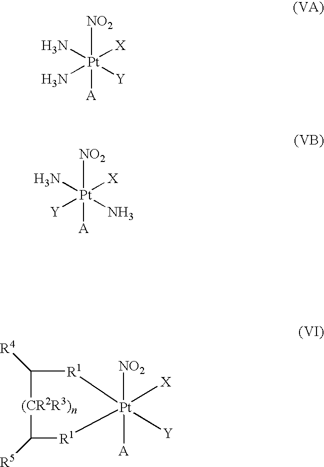
wherein
X and Y are, independently, any halogen, —OH, H₂O, or —SO(CH₃)₂;
or X and Y together form the structure: 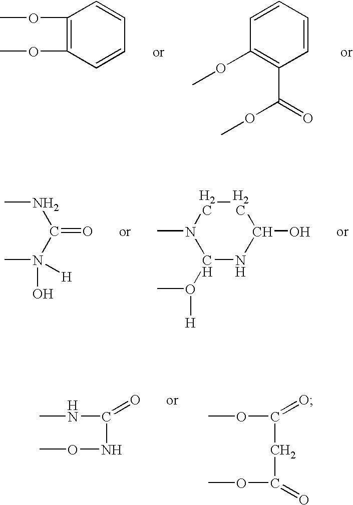
and A can be any of the following: 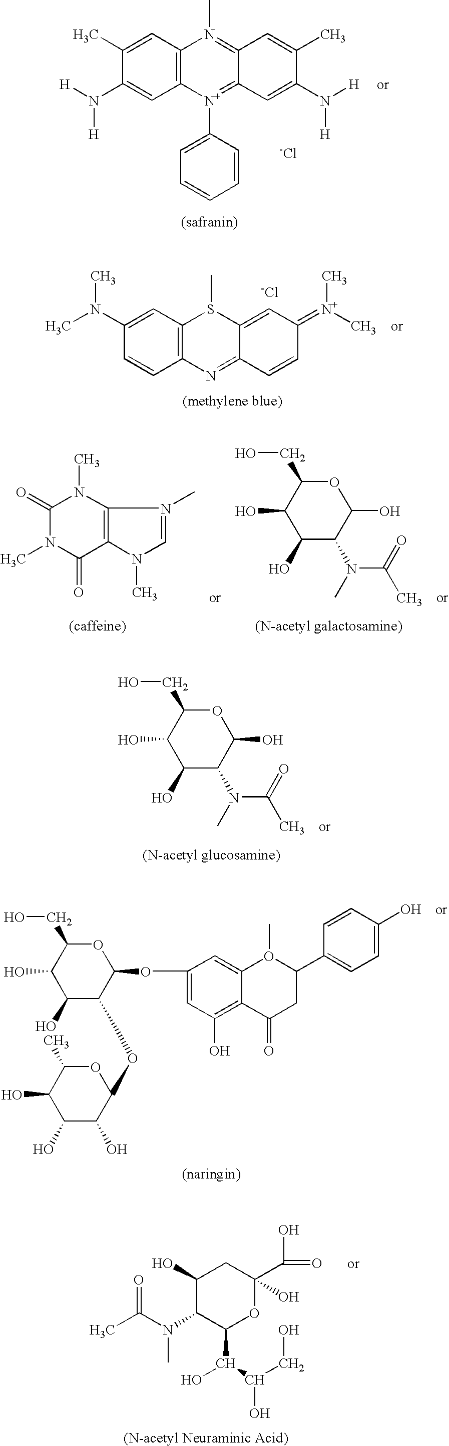 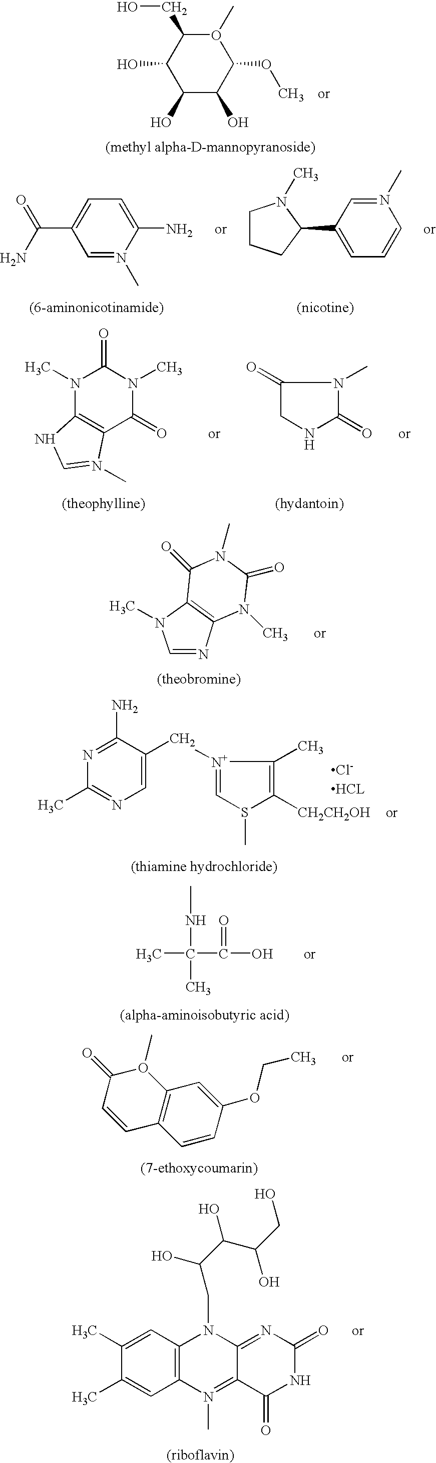 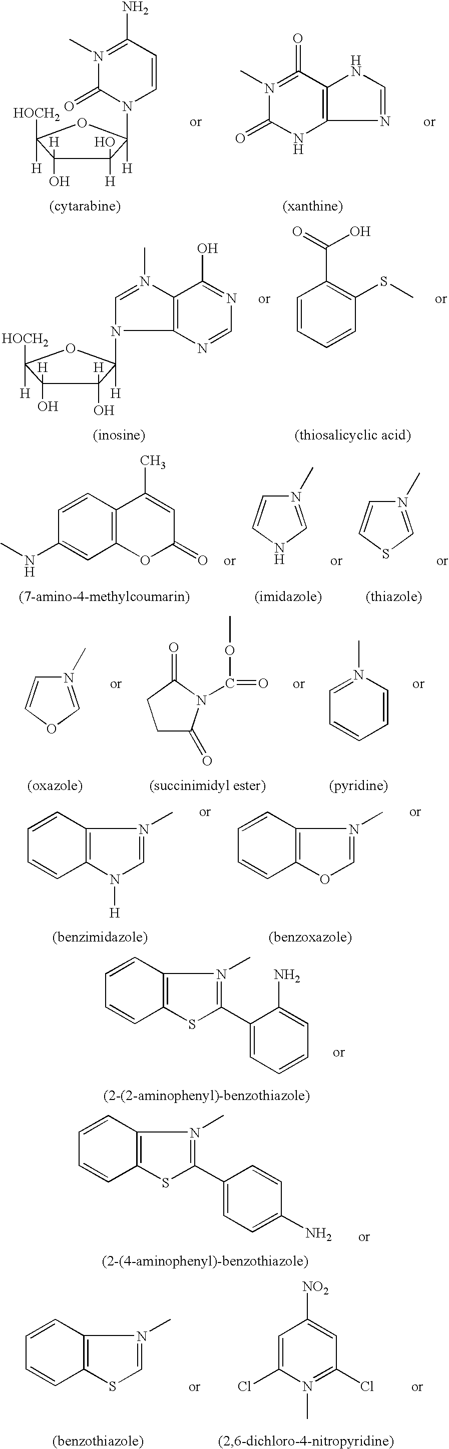 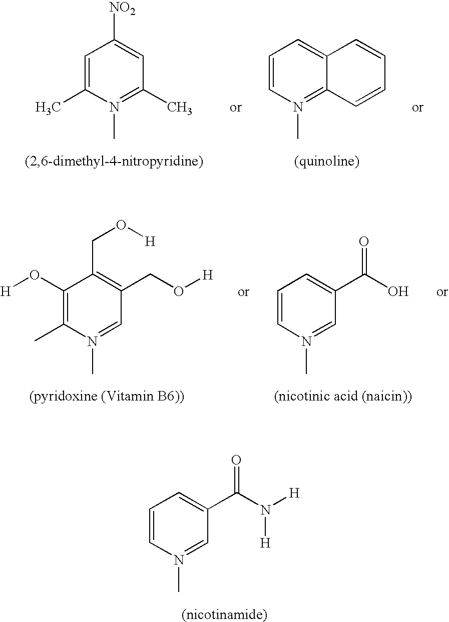
and wherein
R¹ is, independently, NH₂, NH, NR⁴, NHR⁴, N(R⁴)₂, NR⁵, NHR⁵, N(R⁵)₂, or NR⁴R⁵;
R² and R³ are, independently, H, —OH, C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
R⁴ and R⁵ are, independently, H or C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl or R⁴ and R⁵ together form a cycloalkyl, cycloalkoxy, aryl, aryloxy, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
n is any integer from 0 to 6;
or a pharmaceutically acceptable salt thereof; wherein said platinum complex optionally, comprises a detectable label.

147. The kit according to claim 146, wherein said kit further comprises in one or more containers a detectable label that can be coupled, conjugated or bound to said platinum complex.

148. The kit according to claim 146, wherein said kit further comprises in one or more containers a binding moiety that can bind to said platinum complex.

149. The kit according to claim 146, wherein said detectable label is an enzyme, a radioisotope, a chemiluminescent reagent, a bioluminescent reagent, or a fluorescent moiety.

150. A method for assessing metastatic potential of an oncological disorder in a person or animal, or monitoring a person or animal for the onset, progression, or regression of a condition characterized by or associated with abnormal levels of expression of a STAT protein, said method comprising detecting the level of said STAT protein expressed in a cell of said person or animal, wherein the level of said STAT protein expressed in said cell is detected by contacting said cell with a composition comprising:
a) a platinum complex having the structure shown in formula IA or IB: 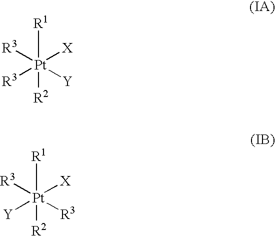
wherein
X and Y are, independently, any halogen, —NO₂, —ONO, or the structure: 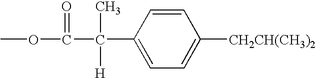
or X and Y together form the structure: 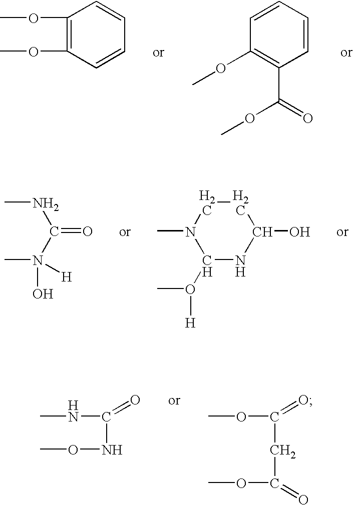
R¹ is —NO₂, —ONO, Cl, Br or F;
R² is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹—NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 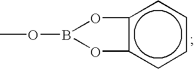
any of which can be substituted with any halogen, —NH₂, —COOH, —OH, alkoxy, cycloalkoxy;
R³ is, independently, —NH₃, —NHR⁷, —NH₂R⁷, —NH(R⁷)₂, or —N(R⁷)₃;
R⁷ is H, C₁₋₆ alkyl, alkoxy, or aryl, any of which can be optionally substituted with any halogen, —NO₂, or —COOH;
R¹⁰ and R¹¹ are, independently, H, —NH₂, —OH, —NHR⁷, —N(R⁷)₂, CONHR⁷, CON(R⁷)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
or a pharmaceutically acceptable salt thereof; or
b) a platinum complex having the structure shown in formula II: 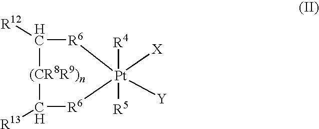
wherein
X and Y are, independently, any halogen, or the structure: 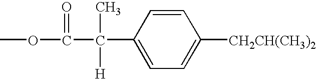
or X and Y together form the structure: 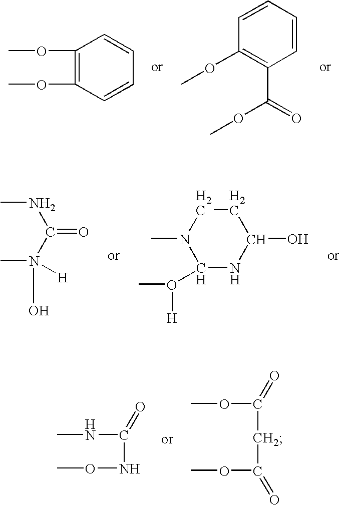
R⁴ is —NO₂ or —ONO;
R⁵ is any halogen, —OH, —ONO, —ONO₂, —COR¹⁰, —OPO₃R¹⁰R¹¹, —OSO₃H, —OSeOOH, —SeOOH, —AsO₂, —OAsO₂, —NR¹⁰R¹¹, —NHR¹⁰R¹¹, —OOCR¹⁵, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, or the structure: 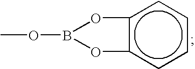
any of which can be substituted with any halogen, —NH₂, —COOH, —OH, or Y and R⁵ form the structure: 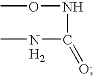
or X and Y together form the structure: 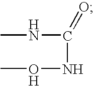
R⁶ is, independently, NH₂, NH, NHR⁷, N(R⁷)₂, NHR⁸, N(R⁸)₂, NHR⁹, N(R⁹)₂, or NR⁸R⁹;
R⁷ is H, C₁₋₆ alkyl, alkoxy, aryl, any of which can be optionally substituted with any halogen, —NO₂, or —COOH;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁰ and R¹¹ are, independently, H, —NH₂, —OH, —NHR⁷, —N(R⁷)₂, CONHR⁷, CON(R)₂, C₁₋₆ alkyl, aryl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H or C₁₋₆ alkyl, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹⁵ is alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with any halogen, —COOH, —OH, —NO₂, —NH₂, —N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof; or
c) a platinum complex having the structure shown in formula III or formula IVA or IVB: 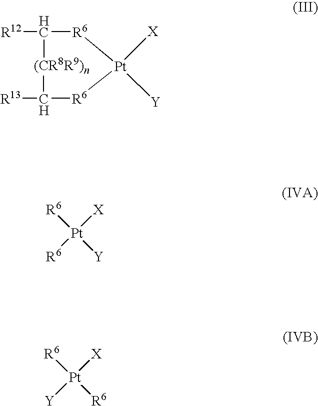
wherein
X and Y are, independently, any halogen, —NO₂, —ONO, or X and Y together form the structure: 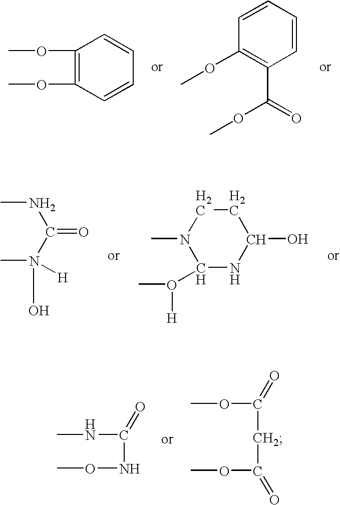
R⁶ is, independently, Cl, Br, F, NO₂, ONO, NHR⁸, NH₂, NHR¹², NR¹², N(R¹²)₂, NHR¹³, NR¹³, N(R¹³)₂, or NR¹²R¹³;
R⁸ and R⁹ are, independently, H, C₁₋₆ alkyl, or —OH, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
R¹² and R¹³ are, independently, H, C₁₋₆ alkyl, or —OH, or R¹² and R¹³ together form an aryl, cycloalkyl, heterocycloalkyl, or heteroaryl, any of which can be optionally substituted with any halogen, —COOH, —OH, —NO₂, —NH₂, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
n is any integer from 0 to 6;
or a pharmaceutically acceptable salt thereof; or
d) a platinum complex having the structure shown in formula VA or VB or formula VI: 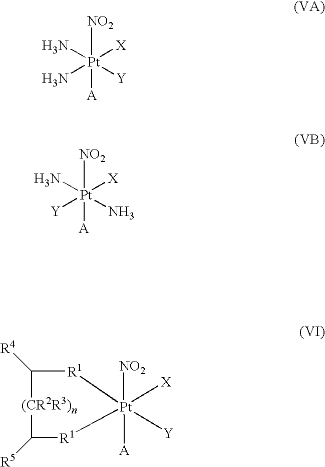
wherein
X and Y are, independently, any halogen, —OH, H₂O, or —SO(CH₃)₂;
or X and Y together form the structure: 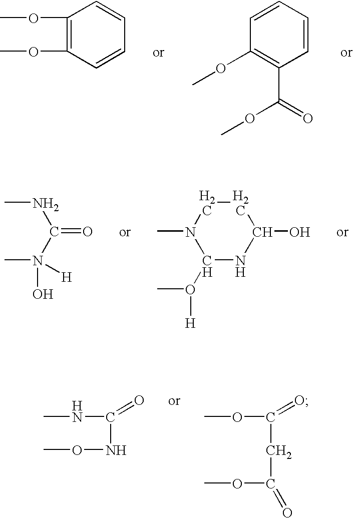
and A can be any of the following: 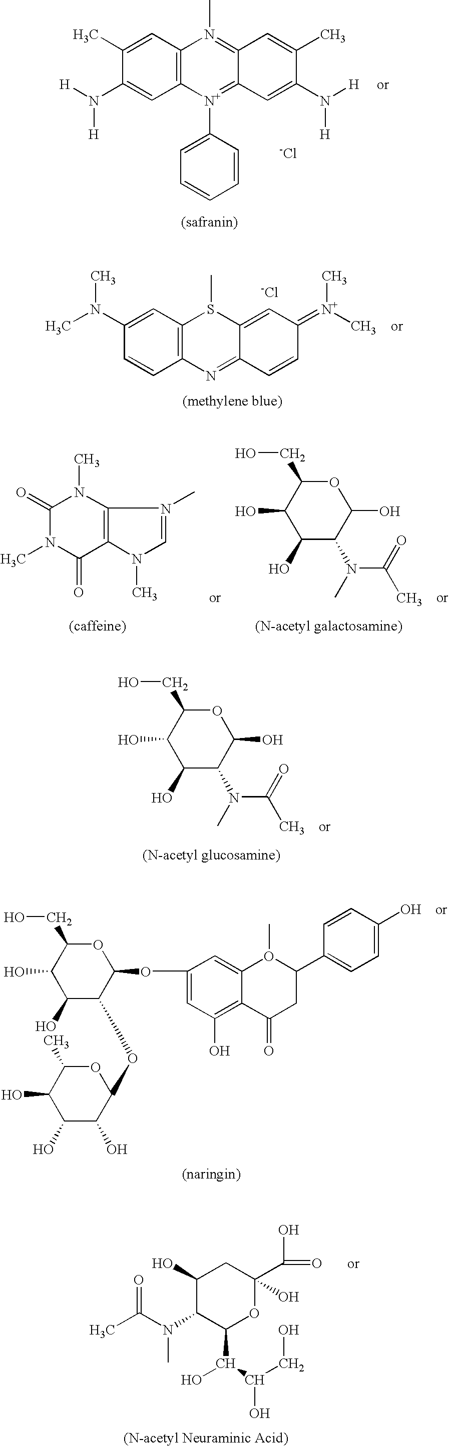 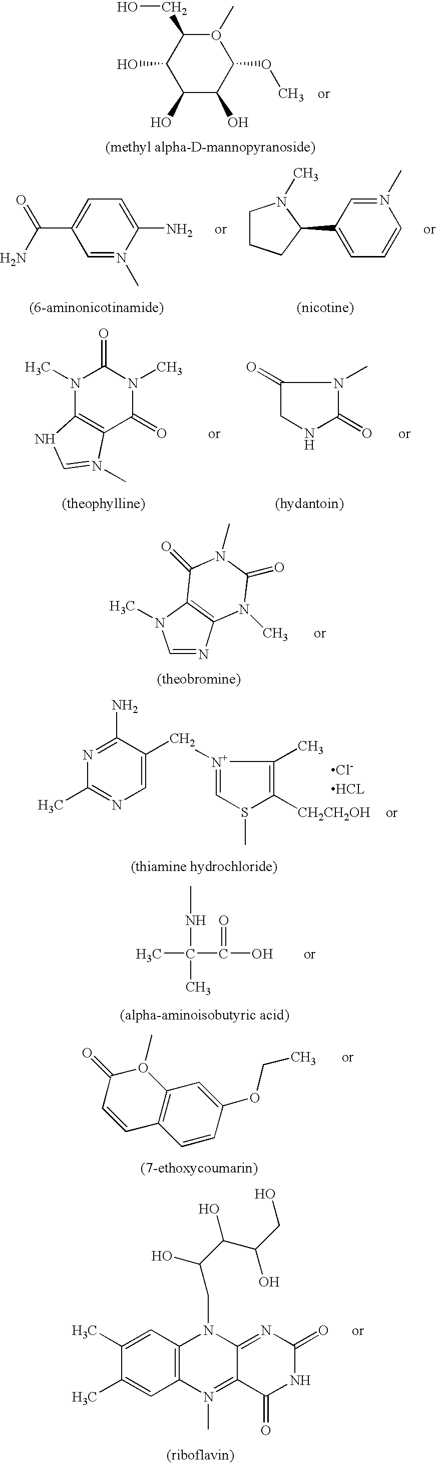 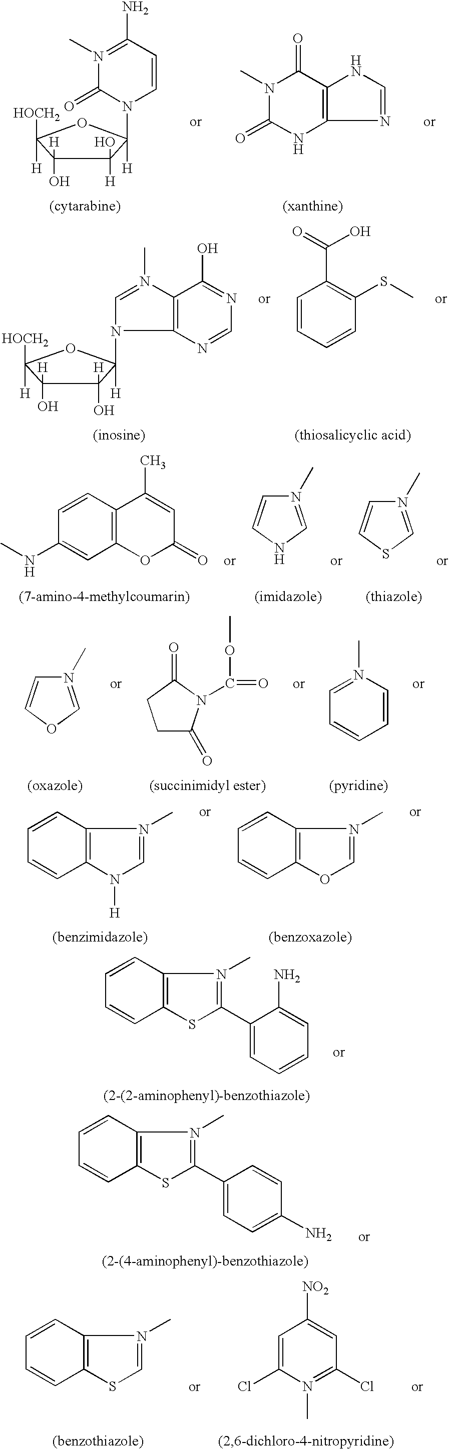 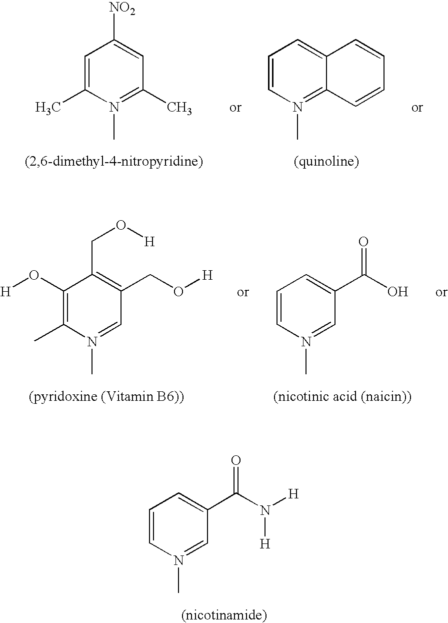
and wherein
R¹ is, independently, NH₂, NH, NR⁴, NHR⁴, N(R⁴)₂, NR⁵, NHR⁵, N(R⁵)₂, or NR⁴R⁵;
R² and R³ are, independently, H, —OH, C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
R⁴ and R⁵ are, independently, H or C₁₋₆ alkyl, alkoxy, cycloalkyl, aryloxy, cycloalkoxy, aryl, heteroalkyl, heterocycloalkyl, heteroaryl, arylcarbonyl, and heteroarylcarbonyl or R⁴ and R⁵ together form a cycloalkyl, cycloalkoxy, aryl, aryloxy, heterocycloalkyl, heteroaryl, arylcarbonyl, and heteroarylcarbonyl, any of which can be optionally substituted with alkyl, alkoxy, cycloalkyl, aryloxy, cycloalkoxy, aryl, heteroalkyl, heterocycloalkyl, heteroaryl, arylcarbonyl, and heteroarylcarbonyl;
n is any integer from 0 to 6;
or a pharmaceutically acceptable salt thereof; and
determining the level of platinum complex in said cell or the level of uptake of said platinum complex into said cell, and correlating said level of said STAT protein expressed in said cell with the metastatic potential of said cell or the onset, progression, or regression of said condition.

151. The method according to claim 150, wherein said condition is an oncological, inflammatory, or neurological disorder.

152. The method according to claim 150, wherein a clinician determines a treatment protocol for said person or animal based upon the assessed metastatic potential of said cell.

153. The method according to claim 150, wherein said oncological disorder is a cancer or tumor of the bone, breast, kidney, mouth, larynx, esophagus, stomach, testis, cervix, head, neck, colon, ovary, lung, bladder, skin, liver, muscle, pancreas, prostate, blood cells (including lymphocytes), or brain.

154. The method according to claim 150, wherein the level of said platinum complex in said cell or the level of uptake of said platinum complex is detected using:
i) an antibody, or an antigen binding fragment thereof, that binds specifically to an epitope of said STAT protein; or
ii) an antibody, or an antigen binding fragment thereof, that binds specifically to an immunogenic or antigenic determinant of or conjugated to said platinum complex.

155. The method according to claim 154, wherein said antibody is directly or indirectly labeled with a detectable label and the presence of said label in said cell is qualitatively or quantitatively detected.

156. The method according to claim 154, wherein said antibody can be detected using a second antibody that binds to said antibody, wherein said second antibody is directly or indirectly labeled with a detectable label and the presence of said label in said cell is qualitatively or quantitatively detected.

157. The method according to claim 150, wherein the level of said platinum complex in said cell or the level of uptake of said platinum complex into said cell is detected using a polypeptide or a peptide that binds specifically to said platinum complex.

158. The method according to claim 157, wherein said polypeptide or peptide that binds specifically to said platinum complex is directly or indirectly labeled with a detectable label and the presence of said label in said cell is qualitatively or quantitatively detected.

159. The method according to claim 157, wherein said peptide or polypeptide that binds specifically to said platinum complex is detected using an antibody, optionally directly or indirectly labeled with a detectable label, that binds specifically to said polypeptide or peptide.

160. The method according to claim 150, wherein the level of said platinum complex in said cell or the level of uptake of said platinum complex into said cell is detected by detection of a substituent or ligand of said platinum complex.

161. The method according to claim 150, wherein the level of said platinum complex in said cell or the level of uptake of said platinum complex into said cell is detected by detecting a detectable label that is coupled or conjugated directly or indirectly to said platinum complex and the presence of said label in said cell is qualitatively or quantitatively detected.
